SEQUENCE LISTING (119 sequences)

SEQ ID NO: 1 - DNA, 18 bp, synthetic construct
  misc_feature 1..18: note = Synthetic Oligonucleotide
  source 1..18: mol_type = other DNA
gatcttgaggctctcatg

SEQ ID NO: 2 - DNA, 20 bp, synthetic construct
  misc_feature 1..20: note = Synthetic Oligonucleotide
  source 1..20: mol_type = other DNA
ataacrttccatggrgccaa

SEQ ID NO: 3 - DNA, 20 bp, synthetic construct
  misc_feature 1..20: note = Synthetic Oligonucleotide
  source 1..20: mol_type = other DNA
ataacrttccatggggccaa

SEQ ID NO: 4 - DNA, 19 bp, synthetic construct
  misc_feature 1..19: note = Synthetic Oligonucleotide
  source 1..19: mol_type = other DNA
ataacgttccatggrgcca

SEQ ID NO: 5 - DNA, 20 bp, synthetic construct
  misc_feature 1..20: note = Synthetic Oligonucleotide
  source 1..20: mol_type = other DNA
ataacgttccatggggccaa

SEQ ID NO: 6 - DNA, 20 bp, synthetic construct
  misc_feature 1..20: note = Synthetic Oligonucleotide
  source 1..20: mol_type = other DNA
cccttagtcagaggtgacag

SEQ ID NO: 7 - DNA, 27 bp, synthetic construct
  misc_feature 1..27: note = Synthetic Oligonucleotide
  source 1..27: mol_type = other DNA
tcaggccccctcaaagccgagatcgcc

SEQ ID NO: 8 - DNA, 26 bp, synthetic construct
  misc_feature 1..26: note = Synthetic Oligonucleotide
  source 1..26: mol_type = other DNA
tcaggccccctcaaagccgaratcgc

SEQ ID NO: 9 - DNA, 26 bp, synthetic construct
  misc_feature 1..26: note = Synthetic Oligonucleotide
  source 1..26: mol_type = other DNA
tcaggccccctcaaagccgagatcgc

SEQ ID NO: 10 - DNA, 26 bp, synthetic construct
  misc_feature 1..26: note = Synthetic Oligonucleotide
  source 1..26: mol_type = other DNA
  modified_base 5..6: mod_base = OTHER; note = uracil
  modified_base 12..14: mod_base = OTHER; note = uracil
  modified_base 24: mod_base = OTHER; note = uracil
gcgatttcggctttgagggggcctga

SEQ ID NO: 11 - DNA, 26 bp, synthetic construct
  misc_feature 1..26: note = Synthetic Oligonucleotide
  source 1..26: mol_type = other DNA
  modified_base 5: mod_base = OTHER; note = uracil
  modified_base 12..14: mod_base = OTHER; note = uracil
  modified_base 24: mod_base = OTHER; note = uracil
gcgatctcggctttgagggggcctga

SEQ ID NO: 12 - DNA, 27 bp, synthetic construct
  misc_feature 1..27: note = Synthetic Oligonucleotide
  source 1..27: mol_type = other DNA
  modified_base 6: mod_base = OTHER; note = uracil
agccattccatgagagcctcaagatcc

SEQ ID NO: 13 - DNA, 26 bp, synthetic construct
  misc_feature 1..26: note = Synthetic Oligonucleotide
  source 1..26: mol_type = other DNA
agccaytccatragagcctcaagatc

SEQ ID NO: 14 - DNA, 26 bp, synthetic construct
  misc_feature 1..26: note = Synthetic Oligonucleotide
  source 1..26: mol_type = other DNA
  modified_base 6: mod_base = OTHER; note = uracil
agccattccatgagagcctcaagatc

SEQ ID NO: 15 - RNA, 26 nt, synthetic construct
  source 1..26: mol_type = other RNA
gatcttgaggctctcatggagtggct

SEQ ID NO: 16 - RNA, 26 nt, synthetic construct
  source 1..26: mol_type = other RNA
gatcttgaggctctcatggaatggct

SEQ ID NO: 17 - RNA, 26 nt, synthetic construct
  source 1..26: mol_type = other RNA
gatcttgaggctcttatggaatggct

SEQ ID NO: 18 - DNA, 24 bp, synthetic construct
  misc_feature 1..24: note = Synthetic Oligonucleotide
  source 1..24: mol_type = other DNA
  modified_base 20: mod_base = OTHER; note = uracil
ctggtgcacttgccagttgtatgc

SEQ ID NO: 19 - DNA, 23 bp, synthetic construct
  misc_feature 1..23: note = Synthetic Oligonucleotide
  source 1..23: mol_type = other DNA
ctggtgcacttgccagttcyatg

SEQ ID NO: 20 - DNA, 23 bp, synthetic construct
  misc_feature 1..23: note = Synthetic Oligonucleotide
  source 1..23: mol_type = other DNA
  modified_base 20: mod_base = OTHER; note = uracil
ctggtgcacttgccagttctatg

SEQ ID NO: 21 - RNA, 23 nt, synthetic construct
  source 1..23: mol_type = other RNA
catagaactggcaagtgcaccag

SEQ ID NO: 22 - RNA, 23 nt, synthetic construct
  source 1..23: mol_type = other RNA
catggaactggcaagtgcaccag

SEQ ID NO: 23 - DNA, 22 bp, synthetic construct
  misc_feature 1..22: note = Synthetic Oligonucleotide
  source 1..22: mol_type = other DNA
cttctaaccgaggtcgaaacgt

SEQ ID NO: 24 - DNA, 20 bp, synthetic construct
  misc_feature 1..20: note = Synthetic Oligonucleotide
  source 1..20: mol_type = other DNA
ataacgttccatggggccaa

SEQ ID NO: 25 - DNA, 19 bp, synthetic construct
  misc_feature 1..19: note = Synthetic Oligonucleotide
  source 1..19: mol_type = other DNA
cccttagtcagaggtgaca

SEQ ID NO: 26 - DNA, 20 bp, synthetic construct
  misc_feature 1..20: note = Synthetic Oligonucleotide
  source 1..20: mol_type = other DNA
cccattctgttgtatatgag

SEQ ID NO: 27 - DNA, 20 bp, synthetic construct
  misc_feature 1..20: note = Synthetic Oligonucleotide
  source 1..20: mol_type = other DNA
cccatcctgttgtatatgag

SEQ ID NO: 28 - DNA, 18 bp, synthetic construct
  misc_feature 1..18: note = Synthetic Oligonucleotide
  source 1..18: mol_type = other DNA
ggtgagcgtgaacacaaa

SEQ ID NO: 29 - DNA, 22 bp, synthetic construct
  misc_feature 1..22: note = Synthetic Oligonucleotide
  source 1..22: mol_type = other DNA
agtggaggatgaagaagatggc

SEQ ID NO: 30 - DNA, 33 bp, synthetic construct
  misc_feature 1..33: note = Synthetic Oligonucleotide
  source 1..33: mol_type = other DNA
gcctgctttgccttctccatcttctgtgcaggc

SEQ ID NO: 31 - DNA, 26 bp, synthetic construct
  misc_feature 1..26: note = Synthetic Oligonucleotide
  source 1..26: mol_type = other DNA
gcctgctttgccttctccatcttctg

SEQ ID NO: 32 - DNA, 34 bp, synthetic construct
  misc_feature 1..34: note = Synthetic Oligonucleotide
  source 1..34: mol_type = other DNA
gcctgctttgccttctccatcttctgtagcaggc

SEQ ID NO: 33 - DNA, 27 bp, synthetic construct
  misc_feature 1..27: note = Synthetic Oligonucleotide
  source 1..27: mol_type = other DNA
gcctgctttgccttctccatcttctgt

SEQ ID NO: 34 - DNA, 38 bp, synthetic construct
  misc_feature 1..38: note = Synthetic Oligonucleotide
  source 1..38: mol_type = other DNA
gcgctagttctgctttgccttctccatcttcctagcgc

SEQ ID NO: 35 - DNA, 31 bp, synthetic construct
  misc_feature 1..31: note = Synthetic Oligonucleotide
  source 1..31: mol_type = other DNA
gcgctagttctgctttgccttctccatcttc

SEQ ID NO: 36 - DNA, 38 bp, synthetic construct; identical to SEQ ID NO: 34
  misc_feature 1..38: note = Synthetic Oligonucleotide
  source 1..38: mol_type = other DNA

SEQ ID NO: 37 - DNA, 31 bp, synthetic construct; identical to SEQ ID NO: 35
  misc_feature 1..31: note = Synthetic Oligonucleotide
  source 1..31: mol_type = other DNA

SEQ ID NO: 38 - DNA, 38 bp, synthetic construct; identical to SEQ ID NO: 34
  misc_feature 1..38: note = Synthetic Oligonucleotide
  source 1..38: mol_type = other DNA

SEQ ID NO: 39 - DNA, 31 bp, synthetic construct; identical to SEQ ID NO: 35
  misc_feature 1..31: note = Synthetic Oligonucleotide
  source 1..31: mol_type = other DNA

SEQ ID NO: 40 - DNA, 34 bp, synthetic construct
  misc_feature 1..34: note = Synthetic Oligonucleotide
  source 1..34: mol_type = other DNA
gctgctagttctgctttgccttctccatcgcagc

SEQ ID NO: 41 - DNA, 29 bp, synthetic construct
  misc_feature 1..29: note = Synthetic Oligonucleotide
  source 1..29: mol_type = other DNA
gctgctagttctgctttgccttctccatc

SEQ ID NO: 42 - DNA, 36 bp, synthetic construct
  misc_feature 1..36: note = Synthetic Oligonucleotide
  source 1..36: mol_type = other DNA
gcctgctagttctgctttgccttctccatcgcaggc

SEQ ID NO: 43 - DNA, 30 bp, synthetic construct
  misc_feature 1..30: note = Synthetic Oligonucleotide
  source 1..30: mol_type = other DNA
gcctgctagttctgctttgccttctccatc

SEQ ID NO: 44 - DNA, 36 bp, synthetic construct; identical to SEQ ID NO: 42
  misc_feature 1..36: note = Synthetic Oligonucleotide
  source 1..36: mol_type = other DNA

SEQ ID NO: 45 - DNA, 30 bp, synthetic construct; identical to SEQ ID NO: 43
  misc_feature 1..30: note = Synthetic Oligonucleotide
  source 1..30: mol_type = other DNA

SEQ ID NO: 46 - DNA, 37 bp, synthetic construct
  misc_feature 1..37: note = Synthetic Oligonucleotide
  source 1..37: mol_type = other DNA
gcctgctagttctgctttgccttctccatcagcaggc

SEQ ID NO: 47 - DNA, 30 bp, synthetic construct; identical to SEQ ID NO: 43
  misc_feature 1..30: note = Synthetic Oligonucleotide
  source 1..30: mol_type = other DNA

SEQ ID NO: 48 - DNA, 37 bp, synthetic construct; identical to SEQ ID NO: 46
  misc_feature 1..37: note = Synthetic Oligonucleotide
  source 1..37: mol_type = other DNA

SEQ ID NO: 49 - DNA, 30 bp, synthetic construct; identical to SEQ ID NO: 43
  misc_feature 1..30: note = Synthetic Oligonucleotide
  source 1..30: mol_type = other DNA

SEQ ID NO: 50 - DNA, 37 bp, synthetic construct; identical to SEQ ID NO: 46
  misc_feature 1..37: note = Synthetic Oligonucleotide
  source 1..37: mol_type = other DNA

SEQ ID NO: 51 - DNA, 30 bp, synthetic construct; identical to SEQ ID NO: 43
  misc_feature 1..30: note = Synthetic Oligonucleotide
  source 1..30: mol_type = other DNA

SEQ ID NO: 52 - DNA, 38 bp, synthetic construct
  misc_feature 1..38: note = Synthetic Oligonucleotide
  source 1..38: mol_type = other DNA
gcctgctagttctgctttgccttctccatctagcaggc

SEQ ID NO: 53 - DNA, 30 bp, synthetic construct; identical to SEQ ID NO: 43
  misc_feature 1..30: note = Synthetic Oligonucleotide
  source 1..30: mol_type = other DNA

SEQ ID NO: 54 - DNA, 34 bp, synthetic construct
  misc_feature 1..34: note = Synthetic Oligonucleotide
  source 1..34: mol_type = other DNA
  modified_base 21: mod_base = OTHER; note = uracil
gcggagaaggcaaagcagaattagcagtctccgc

SEQ ID NO: 55 - DNA, 27 bp, synthetic construct
  misc_feature 1..27: note = Synthetic Oligonucleotide
  source 1..27: mol_type = other DNA
  modified_base 21: mod_base = OTHER; note = uracil
gcggagaaggcaaagcagaattagcag

SEQ ID NO: 56 - DNA, 34 bp, synthetic construct; identical to SEQ ID NO: 54
  misc_feature 1..34: note = Synthetic Oligonucleotide
  source 1..34: mol_type = other DNA
  modified_base 21: mod_base = OTHER; note = uracil

SEQ ID NO: 57 - DNA, 27 bp, synthetic construct
  misc_feature 1..27: note = Synthetic Oligonucleotide
  source 1..27: mol_type = other DNA
  modified_base 21: mod_base = OTHER; note = uracil
gcggagaaggcaaagcagaattagcag

SEQ ID NO: 58 - DNA, 20 bp, synthetic construct
  misc_feature 1..20: note = Synthetic Oligonucleotide
  source 1..20: mol_type = other DNA
tctttcccaccraaccaaca

SEQ ID NO: 59 - DNA, 29 bp, synthetic construct
  misc_feature 1..29: note = Synthetic Oligonucleotide
  source 1..29: mol_type = other DNA
ctagttctgctttgccttctccatcttct

SEQ ID NO: 60 - DNA, 24 bp, synthetic construct
  misc_feature 1..24: note = Synthetic Oligonucleotide
  source 1..24: mol_type = other DNA
aagactcccaccgcagtttcagct

SEQ ID NO: 61 - DNA, 24 bp, synthetic construct
  misc_feature 1..24: note = Synthetic Oligonucleotide
  source 1..24: mol_type = other DNA
aagactcccaccgcagtttcagct

SEQ ID NO: 62 - DNA, 24 bp, synthetic construct
  misc_feature 1..24: note = Synthetic Oligonucleotide
  source 1..24: mol_type = other DNA
aagactcccaccgcagtttcagct

SEQ ID NO: 63 - DNA, 29 bp, synthetic construct
  misc_feature 1..29: note = Synthetic Oligonucleotide
  source 1..29: mol_type = other DNA
ctarttctgctttgccttctccatcttct

SEQ ID NO: 64 - DNA, 29 bp, synthetic construct
  misc_feature 1..29: note = Synthetic Oligonucleotide
  source 1..29: mol_type = other DNA
ctagttctgctttgccttctccatcttct

SEQ ID NO: 65 - DNA, 29 bp, synthetic construct
  misc_feature 1..29: note = Synthetic Oligonucleotide
  source 1..29: mol_type = other DNA
  modified_base 7: mod_base = OTHER; note = uracil
  modified_base 26..27: mod_base = OTHER; note = uracil
agaagatggagaaggcaaagcagaattag

SEQ ID NO: 66 - DNA, 29 bp, synthetic construct
  misc_feature 1..29: note = Synthetic Oligonucleotide
  source 1..29: mol_type = other DNA
  modified_base 7: mod_base = OTHER; note = uracil
  modified_base 27: mod_base = OTHER; note = uracil
agaagatggagaaggcaaagcagaactag

SEQ ID NO: 67 - DNA, 23 bp, synthetic construct
  misc_feature 1..23: note = Synthetic Oligonucleotide
  source 1..23: mol_type = other DNA
gagacacaattgcctacctgctt

SEQ ID NO: 68 - DNA, 26 bp, synthetic construct
  misc_feature 1..26: note = Synthetic Oligonucleotide
  source 1..26: mol_type = other DNA
  modified_base 15: mod_base = OTHER; note = uracil
gagtctaggtcaaattctttcccacc

SEQ ID NO: 69 - DNA, 21 bp, synthetic construct
  misc_feature 1..21: note = Synthetic Oligonucleotide
  source 1..21: mol_type = other DNA
ggtgctcttgaccaaattggg

SEQ ID NO: 70 - DNA, 22 bp, synthetic construct
  misc_feature 1..22: note = Synthetic Oligonucleotide
  source 1..22: mol_type = other DNA
ctttcccaccraaccaacagtg

SEQ ID NO: 71 - DNA, 29 bp, synthetic construct
  misc_feature 1..29: note = Synthetic Oligonucleotide
  source 1..29: mol_type = other DNA
ttagtcatyacagtgactgacaacaaagg

SEQ ID NO: 72 - DNA, 23 bp, synthetic construct
  misc_feature 1..23: note = Synthetic Oligonucleotide
  source 1..23: mol_type = other DNA
aggtaagctccwagatctactat

SEQ ID NO: 73 - DNA, 23 bp, synthetic construct
  misc_feature 1..23: note = Synthetic Oligonucleotide
  source 1..23: mol_type = other DNA
aggtaagctccwagatctactat

SEQ ID NO: 74 - DNA, 23 bp, synthetic construct
  misc_feature 1..23: note = Synthetic Oligonucleotide
  source 1..23: mol_type = other DNA
aggtaagctcctagatctactat

SEQ ID NO: 75 - DNA, 32 bp, synthetic construct
  misc_feature 1..32: note = Synthetic Oligonucleotide
  source 1..32: mol_type = other DNA
  modified_base 9: mod_base = OTHER; note = uracil
ttagtcattacagtgactgacaacaaaggagc

SEQ ID NO: 76 - DNA, 21 bp, synthetic construct
  misc_feature 1..21: note = Synthetic Oligonucleotide
  source 1..21: mol_type = other DNA
tagaccatgtgaattccctgc

SEQ ID NO: 77 - DNA, 29 bp, synthetic construct
  misc_feature 1..29: note = Synthetic Oligonucleotide
  source 1..29: mol_type = other DNA
ttagtcatyacagtgactgacaacaaagg

SEQ ID NO: 78 - DNA, 29 bp, synthetic construct
  misc_feature 1..29: note = Synthetic Oligonucleotide
  source 1..29: mol_type = other DNA
  modified_base 9: mod_base = OTHER; note = uracil
ttagtcattacagtgactgacaacaaagg

SEQ ID NO: 79 - DNA, 26 bp, synthetic construct
  misc_feature 1..26: note = Synthetic Oligonucleotide
  source 1..26: mol_type = other DNA
acaaatgcaaaaatcataccttactc

SEQ ID NO: 80 - DNA, 36 bp, synthetic construct
  misc_feature 1..36: note = Synthetic Oligonucleotide
  source 1..36: mol_type = other DNA
cgtggctttatgtatttgaatgctcctttggccacg

SEQ ID NO: 81 - DNA, 30 bp, synthetic construct
  misc_feature 1..30: note = Synthetic Oligonucleotide
  source 1..30: mol_type = other DNA
cgtggctttatgtatttgaatgctcctttg

SEQ ID NO: 82 - DNA, 36 bp, synthetic construct; identical to SEQ ID NO: 80
  misc_feature 1..36: note = Synthetic Oligonucleotide
  source 1..36: mol_type = other DNA

SEQ ID NO: 83 - DNA, 30 bp, synthetic construct; identical to SEQ ID NO: 81
  misc_feature 1..30: note = Synthetic Oligonucleotide
  source 1..30: mol_type = other DNA

SEQ ID NO: 84 - DNA, 38 bp, synthetic construct
  misc_feature 1..38: note = Synthetic Oligonucleotide
  source 1..38: mol_type = other DNA
cggtggctttatgtatttgaatgctcctttggccaccg

SEQ ID NO: 85 - DNA, 31 bp, synthetic construct
  misc_feature 1..31: note = Synthetic Oligonucleotide
  source 1..31: mol_type = other DNA
cggtggctttatgtatttgaatgctcctttg

SEQ ID NO: 86 - DNA, 39 bp, synthetic construct
  misc_feature 1..39: note = Synthetic Oligonucleotide
  source 1..39: mol_type = other DNA
cggtggctttatgtatttgaatgctcctttgagccaccg

SEQ ID NO: 87 - DNA, 31 bp, synthetic construct; identical to SEQ ID NO: 85
  misc_feature 1..31: note = Synthetic Oligonucleotide
  source 1..31: mol_type = other DNA

SEQ ID NO: 88 - DNA, 26 bp, synthetic construct
  misc_feature 1..26: note = Synthetic Oligonucleotide
  source 1..26: mol_type = other DNA
acaaatgcaaaaatcataccttactc

SEQ ID NO: 89 - DNA, 29 bp, synthetic construct
  misc_feature 1..29: note = Synthetic Oligonucleotide
  source 1..29: mol_type = other DNA
ttagtcattacagtgactgacaacaaagg

SEQ ID NO: 90 - DNA, 29 bp, synthetic construct
  misc_feature 1..29: note = Synthetic Oligonucleotide
  source 1..29: mol_type = other DNA
ttagtcatyacagtgactgacaacaaagg

SEQ ID NO: 91 - DNA, 29 bp, synthetic construct
  misc_feature 1..29: note = Synthetic Oligonucleotide
  source 1..29: mol_type = other DNA
  modified_base 9: mod_base = OTHER; note = uracil
ttagtcattacagtgactgacaacaaagg

SEQ ID NO: 92 - DNA, 23 bp, synthetic construct
  misc_feature 1..23: note = Synthetic Oligonucleotide
  source 1..23: mol_type = other DNA
aggtaagctccgagatctactat

SEQ ID NO: 93 - DNA, 25 bp, synthetic construct
  misc_feature 1..25: note = Synthetic Oligonucleotide
  source 1..25: mol_type = other DNA
ctaggtaagctccaagatctactat

SEQ ID NO: 94 - DNA, 25 bp, synthetic construct
  misc_feature 1..25: note = Synthetic Oligonucleotide
  source 1..25: mol_type = other DNA
ctaggtaagctccaagatctactat

SEQ ID NO: 95 - DNA, 25 bp, synthetic construct
  misc_feature 1..25: note = Synthetic Oligonucleotide
  source 1..25: mol_type = other DNA
ctaggtaagctcctagatctactat

SEQ ID NO: 96 - DNA, 25 bp, synthetic construct
  misc_feature 1..25: note = Synthetic Oligonucleotide
  source 1..25: mol_type = other DNA
ctaggtaagctcctagatctactat

SEQ ID NO: 97 - RNA, 29 nt, synthetic construct
  source 1..29: mol_type = other RNA
cctttgttgtcagtcactgtgatgactaa

SEQ ID NO: 98 - RNA, 29 nt, synthetic construct
  source 1..29: mol_type = other RNA
cctttgttgtcagtcactgtaatgactaa

SEQ ID NO: 99 - DNA, 21 bp, synthetic construct
  misc_feature 1..21: note = Synthetic Oligonucleotide
  source 1..21: mol_type = other DNA
tgaagttgatgaacaaagtgg

SEQ ID NO: 100 - DNA, 23 bp, synthetic construct
  misc_feature 1..23: note = Synthetic Oligonucleotide
  source 1..23: mol_type = other DNA
gatgatgatccygcatcactaac

SEQ ID NO: 101 - DNA, 23 bp, synthetic construct
  misc_feature 1..23: note = Synthetic Oligonucleotide
  source 1..23: mol_type = other DNA
  modified_base 12: mod_base = OTHER; note = uracil
gatgatgatcctgcatcactaac

SEQ ID NO: 102 - DNA, 26 bp, synthetic construct
  misc_feature 1..26: note = Synthetic Oligonucleotide
  source 1..26: mol_type = other DNA
atgggtgcctatgttccagtcatctg

SEQ ID NO: 103 - DNA, 21 bp, synthetic construct
  misc_feature 1..21: note = Synthetic Oligonucleotide
  source 1..21: mol_type = other DNA
caccagccctcaataccaccc

SEQ ID NO: 104 - DNA, 19 bp, synthetic construct
  misc_feature 1..19: note = Synthetic Oligonucleotide
  source 1..19: mol_type = other DNA
gcttcaatggtccacagtt

SEQ ID NO: 105 - DNA, 19 bp, synthetic construct
  misc_feature 1..19: note = Synthetic Oligonucleotide
  source 1..19: mol_type = other DNA
gcttcaatggtccacagtt

SEQ ID NO: 106 - DNA, 23 bp, synthetic construct
  misc_feature 1..23: note = Synthetic Oligonucleotide
  source 1..23: mol_type = other DNA
  modified_base 12: mod_base = OTHER; note = uracil
gatgatgatcctgcatcactaac

SEQ ID NO: 107 - DNA, 34 bp, synthetic construct
  misc_feature 1..34: note = Synthetic Oligonucleotide
  source 1..34: mol_type = other DNA
cgctgctggcacagatgactggaacatagcagcg

SEQ ID NO: 108 - DNA, 28 bp, synthetic construct
  misc_feature 1..28: note = Synthetic Oligonucleotide
  source 1..28: mol_type = other DNA
cgctgctggcacagatgactggaacata

SEQ ID NO: 109 - DNA, 36 bp, synthetic construct
  misc_feature 1..36: note = Synthetic Oligonucleotide
  source 1..36: mol_type = other DNA
ccgagcaagtctgctggcacagatgactgggctcgg

SEQ ID NO: 110 - DNA, 30 bp, synthetic construct
  misc_feature 1..30: note = Synthetic Oligonucleotide
  source 1..30: mol_type = other DNA
ccgagcaagtctgctggcacagatgactgg

SEQ ID NO: 111 - DNA, 38 bp, synthetic construct
  misc_feature 1..38: note = Synthetic Oligonucleotide
  source 1..38: mol_type = other DNA
ccgagcaagtctgctggcacagatgactggttgctcgg

SEQ ID NO: 112 - DNA, 30 bp, synthetic construct; identical to SEQ ID NO: 110
  misc_feature 1..30: note = Synthetic Oligonucleotide
  source 1..30: mol_type = other DNA

SEQ ID NO: 113 - DNA, 33 bp, synthetic construct
  misc_feature 1..33: note = Synthetic Oligonucleotide
  source 1..33: mol_type = other DNA
cgccagtcatctgtgccagcagacttgctggcg

SEQ ID NO: 114 - DNA, 27 bp, synthetic construct
  misc_feature 1..27: note = Synthetic Oligonucleotide
  source 1..27: mol_type = other DNA
cgccagtcatctgtgccagcagacttg

SEQ ID NO: 115 - DNA, 24 bp, synthetic construct
  misc_feature 1..24: note = Synthetic Oligonucleotide
  source 1..24: mol_type = other DNA
tagtatgttgatgcttgcaagttc

SEQ ID NO: 116 - DNA, 984 bp, Influenza A virus
  source 1..984: mol_type = genomic DNA
atgagtcttctaaccgaggtcgaaacgtacgttctttctatcataccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctgaatgggaatggggacccaaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctacaaccaccaatccactaatcaggcatgagaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtacgggtctgaaagatgaccttcttgaaaatttgcaggcctaccagaagcgaatgggagtgcagatgcagcgattcaagtgatcttctcgccattgcagcaaatatcattgggatcttgcacctgatattgtggattactgatcgtctttttttcaaatgtatttatcgtcgctttaaatacggtttgaaaagagggccttctacggaaggagggcctgagtccatgagggaagaatatcaacaggaacagcagagtgctgtggatgttgacgatggtcattttgtcaacatagagctagagtaaaa

SEQ ID NO: 117 - DNA, 1076 bp, Influenza B virus
  source 1..1076: mol_type = genomic DNA
atgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgatatacagaaagcactaattggtgcatctatctgctttttaaaacccaaagaccaggaaagaaaaagaagattcatcacagagcccctatcaggaatgggaacaacagcaacaaaaaagaagggcctgattctagctgagagaaaaatgagaaaatgtgtgagcttccatgaagcatttgaaatagcagaaggccatgaaagctcagcgttactgtattgtctcatggtcatgtacctgaatcctggaaattattcaatgcaagtaaaactaggaacgctctgtgctttgtgcgagaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagaactgcaaagcaacattggagtattgagatctctgggggcaagccaaaagaatggggaaggaattgcaaaggatgtaatggaagtgctaaagcagagctctatgggaaattcagctcttgtgaagaaatacctataatgctcgaaccatttcagattctttcaatttgttcttttattttatcagctctccatttcatggcctggacaataggacatttgaatcaaataaaaagaggagtaaacatgaaaatacgaataaaggggccaaataaagagacgataaacagagaggtatcaattttgagacacaattaccaaaaagaaattcaggctaaagaagcaatgaaggaagtactctctgacaacatggaggtattgagtgaccacatagtaattgaggggctttctgcagaagagataataaaaatgggtgaaacagttttggaggtagaaaaatcgcattaa

SEQ ID NO: 118 - DNA, 15226 bp, respiratory syncytial virus
  source 1..15226: mol_type = genomic DNA
acgcgaaaaaatgcgtacaacaaacttgcgtaaaccaaaaaaatggggcaaataagaatttgataagtaccacttaaatttaactcccttggttagagatgggcagcaattcgttgagtatgataaaagttagattacaaaatttgtttgacaatgatgaagtagcattgttaaaaataacatgctatactgacaaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtgctacaaaatggaggttatatatgggaaatgatggaattaacacattgctctcaacctaatggtctaatagatgacaattgtgaaattaaattctccaaaaaactaagtgattcaacaatgaccaattatatgaatcaattatctgaattacttggatttgatcttaatccataaattataattaatatcaactagcaaatcaatgtcactagcaccattagttaatataaaacttaacagaagacaaaaatggggcaaataaatcaactcagccaacccaaccatggacacaacccacaatgataccacaccacaaagactgatgatcacagacatgagaccgttgtcacttgagactacaataacatcactaaccagagacatcataacacacagatttatatacttaataaatcatgaatgcatagtgagaaaacttgatgaaagacaggccacatttacattcctggtcaactatgaaatgaaactattgcacaaagtaggaagcactaaatataaaaaatatactgaatacaacacaaaatatggcactttccctatgccgatattcatcaatcatgatgggttcttagaatgcattggcattaagcctacaaagcatactcccataatatacaagtatgatctcaatccatgaatttcaacacaagattcacacaatccaaaacaacaactttatgcataactacactccatagtccaaatggagcctgaaaattatagtaatttaaaattaaggagagacataagatagaagatggggcaaatacaaagatggctcttagcaaagtcaagttgaatgatacactcaacaaagatcaacttctgtcatctagcaaatacaccatccaacggagcacaggagatagtattgatactcctaattatgatgtgcagaaacacatcaataagttatgtggcatgttattaatcacagaagatgctaatcataaattcactgggttaataggtatgttatatgctatgtctaggttaggaagagaagacaccataaaaatactcagagatgcgggatatcatgtaaaagcaaatggagtagatgtaacaacacatcgtcaagacatcaatgggaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgattctcctgattgtgggatgataatattatgtatagcagcattagtaataaccaaattggcagcaggggatagatctggtcttacagccgtgattaggagagctaataatgtcctaaaaaatgaaatgaaacgttacaaaggcttactacccaaggatatagccaacagcttctatgaagtgtttgaaaaacatccccactttatagatgtttttgttcattttggtatagcacaatcttccaccagaggtggcagtagagttgaagggatttttgcaggattgtttatgaatgcctatggtgcagggcaagtaatgctacggtggggagtcttagcaaaatcagttaaaaatattatgttaggacatgctagtgtgcaagcagaaatggaacaagttgttgaggtttatgaatatgcccaaaaattgggtggagaagcaggattctaccatatattgaacaacccaaaagcatcattattatctttgactcaatttcctcacttttccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtattagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacctaaagatcccaagaaaaaagatagtatcatatctgtcaactcaatagatatagaagtaaccaaagaaagccctataacatcaaattcaaccattattaacccaacaaatgagacagatgataatgcagggaacaagcccaattatcaaagaaaacctctagtaagtttcaaagaagaccctataccaagtgataatcccttttcaaaactatacaaagaaaccatagagacatttgataacaatgaagaagaatctagctattcatatgaagaaataaatgatcagacgaacgataatataactgcaagattagataggattgatgaaaaattaagtgaaatactaggaatgcttcacacattagtagtagcaagtgcaggacctacatctgctagggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctgttggaagggaatgatagtgacaatgatctatcacttgaagatttctgattagttacaaatctgcacttcaacacacaacaccaacagaagaccaacaaacaaaccaacccactcatccaaccaaacatccatccgccaatcagccaaacagccaacaaaacaaccagccaatccaaaaccagccacctggaaaaaatcgacaatatagttacaaaaaaagaaaagggtggggcaaatatggaaacatacgtgaacaagcttcacgaaggctccacatacacagctgctgttcaatacaatgtcctagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcattgctagcacaaatgcccagcaaatttaccatatgtgctaatgtgtccttggatgaaagaagcaaactggcatatgatgtaaccacaccctgtgaaatcaaggcatgtagtctaacatgcctaaaatcaaaaaatatgttaactacagttaaagatctcactatgaagacactcaaccccacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactattagtcatcacagtgactgacaacaaaggagcattcaaatacataaagccgcaaagtcaattcatagtagatcttggagcttacctagaaaaagaaagtatatattatgttaccacaaattggaagcacacagctacacgatttgcaatcaaacccatggaagattaacctttttcctccacatcagtgagtcaattcatacaaactttctacctacattcttcacttcaccattacaatcacaaacactctgtggttcaaccaatcaaacaaaacttatctgaagtctcagatcatcccaagtcattgttcatcagatctagtaatcaaataagttaataaaaatatacacatggggcaaataatcatcggaggaaatccaactaatcacaatatctgttaacatagacaagtcaacacaccagacagaatcaaccaatggaaaatacatccataacaatagaattctcaagcaaattctggccttactttacactaatacacatgatcacaacaataatctctttgctaatcataatctccatcatgactgcaatactaaacaaactttgtgaatataacgtattccataacaaaacctttgagttaccaagagctcgagtcaacacatagcattcatcaatctaatagctcaaaatagtaaccttgcatttaaaagtgaacaacccccacctctttacaacacctcattaacatcccaccatgcaaaccaccatccatactataaagtagttaattaaaaatagtcataacaatgaactaggatatcaagactaacaataacgttggggcaaatgcaaacatgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtagttattaaaaaacatattatcacaaaaggccatgaccaactcaaacagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaccaagaattagataaatataaaaatgctgtaacagaattgcagttgctcatgcaaagcacaacagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacatcatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaataccagattaacttactatctgtaaaaatgagaactggggcaaatatgtcacgaaggaatccttgcaaatttgaaattcgaggtcattgcttgaatggtaagagatgtcattttagtcataattattttgaatggccaccccatgcactgctcgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaataatataactaaacaatcagcatgtgttgccatgagcaaactcctcactgaactcaatagtgatgatatcaaaaaactgagagacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccacaagagcataaccatcaacaacccaaaagaattaactgttagtgatacaaatgaccatgccaaaaataatgatactacctgacaaatatccttgtagtataacttccatactaataacaagtagatgtagagtcactatgtataatcgaaagaacacactatatttcaatcaaaacaacccaaataaccatatgtactcaccgaatcaaacattcaatgaaatccattggacctcacaagacttgattgacacaattcaaaattttctacagcatctaggtgttattgaggatatatatacaatatatatattagtgtcataacactcaatcctaatactgaccatatcgttgaattattaattcaaataattcaagctgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagagcctacttattttcagtcattacttatgacatacaagagtatgacctcgttggaacagattgctaccactaatttacttaaaaagataataagaagagctatagaaataagtgatgtcaaagtctatgctatattgaataaactagggcttaaagaaaaggacaagattaaatccaacaatggacaggatgaagacaactcagttattacgaccataatcaaagatgatatactttcagctgttaaggataatcaatctcatcttaaagcagacaaaaatcactctacaaaacaaaaagacacaatcaaaacaacactcttgaagaaattaatgtgttcaatgcagcatcctccatcatggttaatacattggtttaatttatacacaaaattaaacaacatattaacacagtatcgatcaaatgaggttaaaaaccatgggtttatattgatagataatcaaactcttagtggatttcaatttattttgaatcaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggtgattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtatccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaagttaattgcaatgagaccaaattttacttgttaagcagtttgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcaccccctaaaaatttgatatggactagtttccctagaaattatatgccgtcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataagagatcatattgtagatcttaacaatgtagatgaacaaagtggattatatagatatcacatgggtggtattgaagggtggtgtcaaaaactatggaccatagaagctatatcactattggatctaatatctctcaaagggaaattctcaattactgctttaattaatggtgacaatcaatcaatagatataagcaaaccagtcagactcatggaaggtcaaactcatgctcaagcagattatttgctagcattaaatagccttaaattactgtataaagagtatgcaggcataggtcacaaattaaaaggaactgagacttatatatcacgagatatgcaatttatgagtaaaacaattcaacataacggtgtatattaccctgctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcaaattgctctacaattaaaaaatcatgcgttatgtaacaataaattatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgatttcctcacagaggctatagttcactctgtgttcatacttagttattatacaaaccatgacttaaaagataaacttcaagatttgtcagatgatagattgaataagttcttaacatgcataatcacgtttgacaaaaaccctaatgctgaattcgtaacattgatgagagatcctcaagctttagggtctgagagacaagctaaaattactagtgaaatcaatagactggcagttacagaggttttgagtacagctccaaacaaaatattctccaaaagtgcacaacattataccactacagagatagatctaaatgatattatgcaaaatatagaacctacatatcctcacgggctaagagttgtttatgaaagtttacccttttataaagcagagaaaatagtaaatcttatatcaggtacaaaatctataactaacatactggaaaagacttctgccatagacttaacagatattgatagagccactgagatgatgaggaaaaacataactttgcttataaggatacttccattggattgtaacagagataaaagagaaatattgagtatggaaaacctaagtattactgaattaagcaaatatgttagggaaagatcttggtctttatccaatatagttggtgttacatcacccagtatcatgtatacaatggacatcaaatatacaacaagcactatagctagtggcataattatagagaaatataatgttaacagtttaacacgtggtgagagaggaccaactaaaccatgggttggttcatctacacaagagaaaaaaacaatgccagtttataatagacaagttttaaccaaaaaacaaagagatcaaatagatctattagcaaaattggattgggtgtatgcatctatagataacaaggatgaattcatggaagaactcagcataggaacccttgggttaacatatgaaaaggccaaaaaattatttccacaatatttaagtgtcaactatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagcttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaacaaaacactcaaatctggatctcatgttaattctaatttaatattggcacataaaatatctgactattttcataatacttacattttaagtactaatttagctggacattggattctaattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaaactggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaatttacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttttacattaattataacttctcagataatactcatctattaactaaacatataaggattgctaattcagaattagaaaataattacaacaaattatatcatcctacaccagaaaccctagagaatatactagccaatccgattaaaagtaatgacaaaaagacactgaacgactattgtataggtaaaaatgttgactcaataatgttaccattgttatctaataagaagcttgttaaatcgtctgcaatgattagaaccaattacagcaaacaagacctgtacaatctattccctacggttgtgatcgatagaattatagatcattcaggtaatacagccaaatccaaccaactttacactactacttcccatcaaatatctttagtgcacaatagcacatcactttattgcatgcttccttggcatcatattaatagattcaattttgtatttagttctacaggttgtaaaattagtatagagtatattttaaaagaccttaaaattaaagatcctaattgtatagcattcataggtgaaggagcagggaatttattattgcgtacagtggtggaacttcatcctgacataagatatatttacagaagtctgaaagattgcaatgatcatagtttacctattgagtttttaaggctatacaatggacatatcaacattgattatggtgaaaatttgaccattcctgctacagatgcaaccaacaacattcattggtcttatttacatataaagtttgctgaacctatcagtctttttgtatgtgatgccgaattgcctgtaacagtcaactggagtaaaattataatagaatggagcaagcatgtaagaaaatgcaagtactgttcctcagttaataaatgtacgttaatagtaaaatatcatgctcaagatgatattgatttcaaattagacaatataactatattaaaaacttatgtatgcttaggcagtaagttaaagggatcggaggtttacttagtccttacaataggtcctgcaaatatatttccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacggaatgaagttttcagcaataaacttataaatcataagcatatgaacatcttaaagtggttcaatcatgttttaaatttcagatcaacagaactaaactataaccatttatatatggtagaatctacatatccttacctaagtgaattgttaaacagcttgacaactaatgaacttaaaaaactgattaaaatcacaggtagtctgttatacaactttcataatgaataatgaataaagatcttataataaaaattcctatagctatacactagcactgtattcaattatagttattaaaaaattaaaaatcatataattttttataaaaataacttttagtgaactaatcctaaagttatcattttgatctaggaggaataaatttaaatcccaatctaattggtttatatgtgtattaactaaactacgagatattagtttttgacactttttttctcgt

SEQ ID NO: 119 - DNA, 15225 bp, respiratory syncytial virus
  source 1..15225: mol_type = genomic DNA
acgcgaaaaaatgcgtactacaaacttgcacattcggaaaaaatggggcaaataagaatttgataagtgctatttaagtctaaccttttcaatcagaaatggggtgcaattcactgagcatgataaaggttagattacaaaatttatttgacaatgacgaagtagcattgttaaaaataacatgttatactgacaaattaattcttctgaccaatgcattagccaaagcagcaatacatacaattaaattaaacggtatagtttttatacatgttataacaagcagtgaagtgtgccctgataacaacattgtagtaaaatctaactttacaacaatgccaatattacaaaacggaggatacatatgggaattgattgagttgacacactgctctcaattaaacggtctaatggatgataattgtgaaatcaaattttctaaaagactaagtgactcagtaatgactaattatatgaatcaaatatctgatttacttgggcttgatctcaattcatgaattatgtttagtctaactcaatagacatgtgtttattaccattttagttaatataaaaactcatcaaagggaaatggggcaaataaactcacctaatcaatcaaactatgagcactacaaatgacaacactactatgcaaagattaatgatcacggacatgagacccctgtcgatggattcaataataacatctctcaccaaagaaatcatcacacacaaattcatatacttgataaacaatgaatgtattgtaagaaaacttgatgaaagacaagctacatttacattcttagtcaattatgagatgaagctactgcacaaagtagggagtaccaaatacaagaaatacactgaatataatacaaaatatggcactttccccatgcctatatttatcaatcatggcgggtttctagaatgtattggcattaagcctacaaaacacactcctataatatacaaatatgacctcaacccgtaaattccaacaaaaaaaaccaacccaaccaaaccaagctattcctcaaacaacaatgctcaatagttaagaaggagctaatccgttttagtaattaaaaataaaagtaaagccaataacataaattggggcaaatacaaagatggctcttagcaaagtcaagttaaatgatacattaaataaggatcagctgctgtcatccagcaaatacactattcaacgtagtacaggagataatattgacactcccaattatgatgtgcaaaaacacctaaacaaactatgtggtatgctattaatcactgaagatgcaaatcataaattcacaggattaataggtatgttatatgctatgtccaggttaggaagggaagacactataaagatacttaaagatgctggatatcatgttaaagctaatggagtagatataacaacatatcgtcaagatataaatggaaaggaaatgaaattcgaagtattaacattatcaagcttgacatcagaaatacaagtcaatattgagatagaatctagaaaatcctacaaaaaaatgctaaaagagatgggagaagtggctccagaatataggcatgattctccagactgtgggatgataatactgtgtatagcagcacttgtaataaccaaattagcagcaggagacagatcaggtcttacagcagtaattaggagggcaaacaatgtcttaaaaaatgaaataaaacgctacaagggtctcataccaaaggatatagctaacagtttttatgaagtgtttgaaaaacaccctcatcttatagatgtttttgtgcactttggcattgcacaatcatcaacaagagggggtagtagagttgaaggaatctttgcaggattgtttatgaatgcctatggttcagggcaagtaatgctaagatggggagttttagccaaatctgtaaaaaatatcatgctaggtcatgctagtgtccaggcagaaatggagcaagttgtggaagtctatgagtatgcacagaagttgggaggagaagctggattctaccatatattgaacaatccaaaagcatcattgctgtcattaactcaatttcctaacttctcaagtgtggtcctaggcaatgcagcaggtctaggcataatgggagagtatagaggtacgccaagaaaccaggatctttatgatgcagccaaagcatatgcagagcaactcaaagaaaatggagtaataaactacagtgtattagacttaacagcagaagaattggaagccataaagaatcaactcaaccctaaagaagatgatgtagagctttaagttaacaaaaaatacggggcaaataagtcaacatggagaagtttgcacctgaatttcatggagaagatgcaaataacaaagctaccaaattcctagaatcaataaagggcaagttcgcatcatccaaagatcctaagaagaaagatagcataatatctgttaactcaatagatatagaagtaaccaaagagagcccgataacatctggcaccaacatcatcaatccaacaagtgaagccgacagtaccccagaaaccaaagccaactacccaagaaaacccctagtaagcttcaaagaagatctcaccccaagtgacaaccctttttctaagttgtacaaagaaacaatagaaacatttgataacaatgaagaagaatctagctactcatatgaagagataaatgatcaaacaaatgacaacattacagcaagactagatagaattgatgaaaaattaagtgaaatattaggaatgctccatacattagtagttgcaagtgcaggacccacttcagctcgcgatggaataagagatgctatggttggtctgagagaagaaatgatagaaaaaataagagcggaagcattaatgaccaatgataggttagaggctatggcaagacttaggaatgaggaaagcgaaaaaatggcaaaagacacctcagatgaagtgcctcttaatccaacttccaaaaaattgagtgacttgttggaagacaacgatagtgacaatgatctgtcacttgatgatttttgatcagtgatcaactcactcagcaatcaacaacatcaataaaacagacatcaatccattgaatcaactgccagaccgaacaaacaaatgtccgtcagcggaaccaccaaccaatcaatcaaccaactgatccatcagcaacctgacgaaattaacaatatagtaacaaaaaaagaacaagatggggcaaatatggaaacatacgtgaacaagcttcacgaaggctccacatacacagcagctgttcagtacaatgttctagaaaaagatgatgatcctgcatcactaacaatatgggtgcctatgttccagtcatctgtaccagcagacttgctcataaaagaacttgcaagcatcaacatactagtgaagcagatctctacgcccaaaggaccttcactacgagtcacgattaactcaagaagtgctgtgctggctcaaatgcctagtaatttcatcataagcgcaaatgtatcattagatgaaagaagcaaattagcatatgatgtaactacaccttgtgaaatcaaagcatgcagtctaacatgcttaaaagtgaaaagtatgttaactacagtcaaagatcttaccatgaagacattcaaccccactcatgagatcattgctctatgtgaatttgaaaatattatgacatcaaaaagagtaataataccaacctatctaagaccaattagtgtcaaaaacaaggatctgaactcactagaaaacatagcaaccaccgaattcaaaaatgctatcaccaatgcgaaaattattccctatgctggattagtattagttatcacagttactgacaataaaggagcattcaaatatatcaagccacagagtcaatttatagtagatcttggtgcctacctagaaaaagagagcatatattatgtgactactaattggaagcatacagctacacgtttttcaatcaaaccactagaggattaaatttaattatcaacactgaatgacaggtccacatatatcctcaaactacacactatatccaaacatcatgaacatctacactacacacttcatcacacaaaccaatcccactcaaaatccaaaatcactaccagccactatctgctagacctagagtgcgaataggtaaataaaaccaaaatatggggtaaatagacattagttagagttcaatcaatctcaacaaccatttataccgccaattcaatacatatactataaatcttaaaatgggaaatacatccatcacaatagaattcacaagcaaattttggccctattttacactaatacatatgatcttaactctaatctctttactaattataatcactattatgattgcaatactaaataagctaagtgaacataaaacattctgtaacaatactcttgaactaggacagatgcatcaaatcaacacatagtgctctaccatcatgctgtgtcaaattataatcctgtatatataaacaaacaaatccaatcttctcacagagtcatggtgtcgcaaaaccacgccaactatcatggtagcatagagtagttatttaaaaattaacataatgatgaattattagtatgggatcaaaaacaacattggggcaaatgcaaccatgtccaaacacaagaatcaacgcactgccaggactctagaaaagacctgggatactctcaatcatctaattgtaatatcctcttgtttatacagattaaatttaaaatctatagcacaaatagcactatcagttctggcaatgataatctcaacctctctcataattgcagccataatattcatcatctctgccaatcacaaagttacactaacaacggtcacagttcaaacaataaaaaaccacactgaaaaaaacatcaccacctaccttactcaagtcccaccagaaagggttagctcatccaaacaacctacaaccacatcaccaatccacacaaattcagccacaacatcacccaacacaaagtcagaaacacaccacacaacagcacaaaccaaaggcagaaccaccacctcaacacagaccaacaagccgagcacaaaaccacgcctaaaaaatccaccaaaaaaaccaaaagatgattaccattttgaagtgttcaacttcgttccctgtagtatatgtggcaacaatcaactttgcaaatccatctgtaaaacaataccaagcaacaaaccaaagaagaaaccaaccatcaaacccacaaacaaaccaaccaccaaaaccacaaacaaaagagacccaaaaacaccagccaaaacgacgaaaaaagaaactaccaccaacccaacaaaaaaaccaaccctcacgaccacagaaagagacaccagcacctcacaatccactgtgctcgacacaaccacattagaacacacaatccaacagcaatccctccactcaaccacccccgaaaacacacccaactccacacaaacacccacagcatccgagccctctacatcaaattccacccaaaatacccaatcacatgcttagttattcaaaaactacatcttagcagaaaaccgtgacctatcaagcaagaacgaaattaaacctggggcaaataaccatggagctgctgatccacaggttaagtgcaatcttcctaactcttgctattaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtgacagaattacagctacttatgcaaaacacaccagctgccaacaaccgggccagaagagaagcaccacagtatatgaactatacaatcaataccactaaaaacctaaatgtatcaataagcaagaagaggaaacgaagatttctgggcttcttgttaggtgtaggatctgcaatagcaagtggtatagctgtatccaaagttctacaccttgaaggagaagtgaacaagatcaaaaatgctttgttatctacaaacaaagctgtagtcagtctatcaaatggggtcagtgttttaaccagcaaagtgttagatctcaagaattacataaataaccaattattacccatagtaaatcaacagagctgtcgcatctccaacattgaaacagttatagaattccagcagaagaacagcagattgttggaaatcaacagagaattcagtgtcaatgcaggtgtaacaacacctttaagcacttacatgttaacaaacagtgagttactatcattgatcaatgatatgcctataacaaatgatcagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttctttccacaggctgacacttgtaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctcttggagctatagtgtcatgctatggtaaaactaaatgcactgcatccaacaaaaatcgtgggattataaagacattttctaatggttgtgactatgtgtcaaacaaaggagtagatactgtgtcagtgggcaacactttatactatgtaaacaagctggaaggcaagaacctttatgtaaaaggggaacctataataaattactatgaccctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttgctgttgtattgcaaagccaaaaacacaccagttacactaagcaaagaccaactaagtggaatcaataatattgcattcagcaaatagacaaaaaaccacctgatcatgtttcaacaacagtctgctgatcaccaatcccaaatcaacccataacaaacacttcaacatcacagtacaggctgaatcatttcttcacatcatgctacccacacaactaagctagatccttaactcatagttacataaaaacctcaagtatcacaatcaaacactaaatcaacacatcattcacaaaattaacagctggggcaaatatgtcgcgaagaaatccttgtaaatttgagattagaggtcattgcttgaatggtagaagatgtcactacagtcataattactttgaatggcctcctcatgccttactagtgaggcaaaacttcatgttaaacaagatactcaagtcaatggacaaaagcatagacactttgtctgaaataagtggagctgctgaactggacagaacagaagaatatgctcttggtatagttggagtgctagagagttacataggatctataaacaacataacaaaacaatcagcatgtgttgctatgagtaaacttcttattgagatcaatagtgatgacattaaaaagctgagagataatgaagaacccaattcacctaagataagagtgtacaatactgttatatcatacattgagagcaatagaaaaaacaacaagcaaacaatccatctgctcaaaagactaccagcagacgtgctgaagaagacaataaaaaacacattagatatccacaaaagcataatcataagcaacccaaaagagtcaaccgtgaatgatcaaaatgaccaaaccaaaaataatgatattaccggataaatatccttgtagtatatcatccatattgatttcaagtgaaagcatgattgctacattcaatcataaaaacatattacaatttaaccataaccatttggataaccaccagcgtttattaaataatatatttgatgaaattcattggacacctaaaaacttattagatgccactcaacaatttctccaacatcttaacatccctgaagatatatatacaatatatatattagtgtcataatgcttggccataacgattctatatcatccaaccataaaactatcttaataaggttatgggacaaaatggatcccattattaatggaaactctgctaatgtgtatctaactgatagttatttaaaaggtgttatctctttttcagaatgtaatgctttagggagttacctttttaacggcccttatctcaaaaatgattacaccaacttaattagtagacaaagtccactactagagcatatgaatcttaaaaaactaactataacacagtcattaatatctagatatcataaaggtgaactgaaattagaagaaccaacttatttccagtcattacttatgacatataaaagcatgtcctcgtctgaacaaattgctacaactaacttacttaaaaaaataatacgaagagctatagaaataagtgatgtaaaggtgtacgccatcttgaataaactaggactaaaggaaaaggacagagttaagcccaacaataattcaggtgatgaaaactcagtacttacaactataattaaagatgatatactttcggctgtggaaagcaatcaatcatatacaaattcagacaaaaatcactcagtaaatcaaaatatcactatcaaaacaacactcttgaaaaaattgatgtgttcaatgcaacatcctccatcatggttaatacactggttcaatttatatacaaaattaaataacatattaacacaatatcgatcaaatgaggtaaaaagtcatgggtttatattaatagataatcaaactttaagtggttttcagtttattttaaatcaatatggttgtatcgtttatcataaaggactcaaaaaaatcacaactactacttacaatcaatttttaacatggaaagacatcagccttagcagattaaatgtttgcttaattacttggataagtaattgtttgaatacattaaataaaagcttagggctgagatgtggattcaataatgttgtgttatcacaattatttctttatggagattgtatactgaaattatttcataatgaaggcttctacataataaaagaagtagagggatttattatgtctttaattctaaacataacagaagaagatcaatttaggaaacgattttataatagcatgctaaataacatcacagatgcagctattaaggctcaaaagaacctactatcaagggtatgtcacactttattagacaagacagtgtctgataatatcataaatggtaaatggataatcctattaagtaaatttcttaaattgattaagcttgcaggtgataataatctcaataatttgagtgagctatattttctcttcagaatctttggacatccaatggttgatgaaagacaagcaatggatgctgtaagaattaactgtaatgaaactaagttctacttattaagtagtctaagtacgttaagaggtgctttcatttatagaatcataaaagggtttgtaaatacctacaacagatggcccactttaaggaatgctattgtcctacctctaagatggttaaactattataaacttaatacttatccatctctacttgaaatcacagaaaatgatttgattattttatcaggattgcggttctatcgtgaatttcatctgcctaaaaaagtggatcttgaaatgataataaatgacaaagccatttcacctccaaaagatctaatatggactagttttcctagaaattacatgccatcacatatacaaaattatatagaacatgaaaagttgaagttctctgaaagcgacagatcaagaagagtactagagtattacttgagagataataaattcaatgaatgcgatctatacaattgtgtagtcaatcaaagctatctcaacaactctaatcacgtggtatcactaactggtaaagaaagagagctcagtgtaggtagaatgtttgctatgcaaccaggtatgtttaggcaaatccaaatcttagcagagaaaatgatagccgaaaatattttacaattcttccctgagagtttgacaagatatggtgatctagagcttcaaaagatattagaattaaaagcaggaataagcaacaagtcaaatcgttataatgataactacaacaattatatcagtaaatgttctatcattacagatcttagcaaattcaatcaagcatttagatatgaaacatcatgtatctgcagtgatgtattagatgaactgcatggagtacaatctctgttctcttggttgcatttaacaatacctcttgtcacaataatatgtacatatagacatgcacctcctttcataaaggatcatgttgttaatcttaatgaagttgatgaacaaagtggattatacagatatcatatgggtggtattgagggctggtgtcaaaaactgtggaccattgaagctatatcattattagatctaatatctctcaaagggaaattctctatcacagctctgataaatggtgataatcagtcaattgatataagtaaaccagttagacttatagagggtcagacccatgctcaagcagattatttgttagcattaaatagccttaaattgctatataaagagtatgcaggtataggccataagcttaagggaacagagacctatatatcccgagatatgcagttcatgagcaaaacaatccagcacaatggagtgtactatccagccagtatcaaaaaagtcctgagagtaggtccatggataaatacaatacttgatgattttaaagttagtttagaatctataggtagcttaacacaggagttagaatacagaggggaaagcttattatgcagtttaatatttaggaacatttggttatacaatcaaattgctttgcaactccgaaatcatgcattatgtaacaataagctatatttagatatattgaaagtattaaaacacttaaaaactttttttaatcttgatagtatcgatatggcgttatcattgtatatgaatttgcctatgctgtttggtggtggtgatcctaatttgttatatcgaagcttttataggagaactccagacttccttacagaagctatagtacattcagtgtttgtgttgagctattatactggtcacgatttacaagataagctccaggatcttccagatgatagactgaacaaattcttgacatgtgtcatcacattcgataaaaatcccaatgccgagtttgtaacattgatgagggatccacaggcgttagggtctgaaaggcaagctaaaattactagtgagattaatagattagcagtaacagaagtcttaagtatagctccaaacaaaatattttctaaaagtgcacaacattatactaccactgagattgatctaaatgacattatgcaaaatatagaaccaacttaccctcatggattaagagttgtttatgaaagtctacctttttataaagcagaaaaaatagttaatcttatatcaggaacaaaatccataactaatatacttgaaaaaacatcagcaatagatacaactgatattaatagggctactgatatgatgaggaaaaatataactttacttataaggatacttccactagattgtaacaaagacaaaagagagttattaagtttagaaaatcttagtataactgaattaagcaagtatgtaagagaaagatcttggtcattatccaatatagtaggagtaacatcgccaagtattatgttcacaatggacattaaatatacaactagcactatagccagtggtataattatagaaaaatataatgttaatagtttaactcgtggtgaaagaggacctactaagccatgggtaggttcatctacgcaggagaaaaaaacaatgccagtgtacaatagacaagttttaaccaaaaagcaaagagaccaaatagatttattagcaaaattagactgggtatatgcatccatagacaacaaagatgaattcatggaagaactgagtactggaacacttggactgtcatatgaaaaagccaaaaagttgtttccacaatatctaagtgtcaattatttacaccgtttaacagtcagtagtagaccatgtgaattccctgcatcaataccagcttatagaacaacaaattatcatttcgatactagtcctatcaatcatgtattaacagaaaagtatggagatgaagatatcgacattgtgtttcaaaattgcataagttttggtcttagcctgatgtcggttgtggaacaattcacaaacatatgtcctaatagaattattctcataccgaagctgaatgagatacatttgatgaaacctcctatatttacaggagatgttgatatcatcaagttgaagcaagtgatacaaaaacagcatatgttcctaccagataaaataagtttaacccaatatgtagaattattcctaagtaacaaagcacttaaatctggatctaacatcaattctaatttaatattagtacataaaatgtctgattattttcataatgcttatattttaagtactaatttagctggacattggattctaattattcaacttatgaaagattcaaaaggtatttttgaaaaagattggggagaggggtacataactgatcatatgttcattaatttgaatgttttctttaatgcttataagacttatttgctatgttttcataaaggttatggtaaagcaaaattagaatgtgatatgaacacttcagatcttctttgtgttttggagttaatagacagtagctactggaaatctatgtctaaagttttcctagaacaaaaagtcataaaatacatagtcaatcaagacacaagtttgcatagaataaaaggctgtcacagttttaagttgtggtttttaaaacgccttaataatgctaaatttaccgtatgcccttgggttgttaacatagattatcacccaacacatatgaaagctatattatcttacatagatttagttagaatggggttaataaatgtagataaattaaccattaaaaataaaaacaaattcaatgatgaattttacacatcaaatctcttttacattagttataacttttcagacaacactcatttgctaacaaaacaaataagaattgctaattcagaattagaagataattataacaaactatatcacccaaccccagaaactttagaaaatatatcattaattcctgttaaaagtaataatagtaacaaacctaaattttgtataagtggaaataccgaatctataatgatgtcaacattctctaataaaatgcatattaaatcttccactgttaccacaagattcaattatagcaaacaagacttgtacaatttatttccaaatgttgtgatagacaggattatagatcattcaggtaatacagcaaaatctaaccaactttacatcaccacttcacatcagacatctttagtaaggaatagtgcatcactttattgcatgcttccttggcatcatgtcaatagatttaactttgtatttagttccacaggatgcaagatcagtatagagtatattttaaaagatcttaagattaaggaccccagttgtatagcattcataggtgaaggagctggtaacttattattacgtacggtagtagaacttcatccagacataagatacatttacagaagtttaaaagattgcaatgatcatagtttacctattgaatttctaagattatacaacgggcatataaacatagattatggtgagaatttaaccattcctgctacagatgcaactaataacattcattggtcttatttacatataaaatttgcagaacctattagcatctttgtctgcgatgctgaattacctgttacagccaattggagtaaaattataattgaatggagtaagcatgtaagaaagtgcaagtactgttcttctgtaaatagatgcattttaatcgcaaaatatcatgctcaagatgatattgatttcaaattagataacattactatattaaaaacttacgtgtgcctaggtagcaagttaaaaggatctgaagtttacttagtccttacaataggccctgcaaatatacttcctgtttttgatgttgtgcaaaatgctaaattgattttttcaagaactaaaaatttcattatgcctaaaaaaactgacaaggaatctatcgatgcaaatattaaaagcttaatacctttcctttgttaccctataacaaaaaaaggaattaagacttcattgtcaaaattgaagagtgtagttaatggggatatattatcatattctatagctggacgtaatgaagtattcagcaacaagcttataaaccacaagcatatgaatatcctaaaatggctagatcatgttttaaattttagatcagctgaacttaattacaatcatttatacatgatagagtccacatatccttacttaagtgaattgttaaatagtttaacaaccaatgagctcaagaaactgattaaaataacaggtagtgtactatacaaccttcccaacgaacagtaacttaaaatatcattaacaagtttggtcaaatttagatgctaacacatcattatattatagttattaaaaaatatgcaaacttttcaataatttagcttactgattccaaaattatcattttatttttaaggggttgaataaaagtctaaaactaacaatgatacatgtgcatttacaacacaacgagacattagtttttgacactttttttctcgt

(19) United States
(12) Patent Application Publication
(10) Pub. No.: US 2024/0247325 A1
(43) Pub. Date: Jul. 25, 2024

(54) COMPOSITIONS AND METHODS FOR DETECTION OF VIRAL PATHOGENS IN SAMPLES

(71) Applicant: Gen-Probe Incorporated, San Diego, CA (US)

(72) Inventors: Matthias Jost, San Diego, CA (US); Pamela Douglass, Kansas City, MO (US); Daniel P. Kolk, Ramona, CA (US); Mehrdad R. Majlessi, Escondido, CA (US)

(21) Appl. No.: 18/625,400

(22) Filed: Apr. 3, 2024

Related U.S. Application Data
(62) Division of application No. 16/488,909, filed on Aug. 26, 2019, now Pat. No. 11,976,337.
(60) Provisional application No. 62/476,659, filed on Mar. 24, 2017.

(51) Int. Cl. C12Q 1/70

(52) U.S. Cl. CPC C12Q 1/701; C12Q 1/70; C12Q 2561/101; C12Q 2563/107; C12Q 2600/16

(57) ABSTRACT

This disclosure concerns amplification primers, hybridization assay probes, compositions containing such primers and probes, and associated reagents, kits, and methods, that can be used to analyze samples for the presence of Influenza A virus, Influenza B virus, Respiratory Syncytial Virus A, and/or Respiratory Syncytial Virus B target nucleic acids.

### CROSS REFERENCE TO RELATED APPLICATIONS

This application is a divisional of U.S. Provisional application Ser. No. 16/488,909, filed Aug. 26, 2019, which is a national stage entry of PCT/US2018/023995, filed Mar. 23, 2018, which claims priority under 35 U.S.C. § 119 of U.S. Provisional Application 62/476,659, filed Mar. 24, 2017, the contents of each of which are incorporated herein by reference.

### SEQUENCE LISTING

The Sequence Listing written in file DIA.0049_SeqList-ST26.xml is 65 kilobytes in size, was created Apr. 2, 2024, and is hereby incorporated by reference.

### BACKGROUND

### Field

The present disclosure relates to the field of biotechnology. More specifically, the disclosure relates to compositions, including kits and reagents, and methods for analysis of samples to detect viral pathogens, particularly Influenza Virus and Respiratory Syncytial Virus.

### Background

Influenza is an acute respiratory illness in humans caused by infection with the Influenza (Flu) virus, primarily types A and B. Influenza A viruses are further categorized into subtypes based on two major surface protein antigens, hemagglutinin (H), and neuraminidase (N). Influenza B viruses are not categorized into subtypes. The Influenza viruses are RNA viruses in the family Orthomyxoviridae. Each of Influenza types A and B (Flu A and Flu B, respectively) is a separate genus containing one species and a large number of sub-species.

Influenza epidemics occur yearly around the world. Although both Flu types A and B circulate in the population, type A is usually dominant. These yearly epidemics are partly due to antigenic variation in the H and N surface proteins of the virus. Transmission of influenza is primarily via airborne droplet (coughing or sneezing). Symptoms arise on average 1 to 2 days post-exposure and include fever, chills, headache, malaise, cough, and coryza. Gastrointestinal symptoms such as nausea, vomiting, and diarrhea can occur, primarily in young children. Complications due to influenza include pneumonia, which can cause increased morbidity and mortality in pediatric, elderly, and immune-compromised populations. In the United States, it is estimated that influenza results in more than 200,000 hospitalizations and up to 36,000 deaths annually. Large influenza outbreaks, or pandemics, occur rarely. In the 20^{th} Century, three influenza pandemics occurred, in 1918, 1958, and 1968, each causing millions of deaths worldwide. Influenza may also affect other animals, including pigs, horses and birds.

Respiratory syncytial virus (RSV) is the leading cause of lower respiratory tract infections in infants and children. Like Influenza, RSV is an RNA virus. RSV is a member of the family Paramyxoviridae, in the genus Orthopneumovirus. There are 2 types of RSV, A and B, which are differentiated based on antigenic and surface protein variations. Most yearly epidemics contain a mix of RSV A and RSV B, but one subgroup can dominate during a season. RSV infection can cause severe respiratory illness among all ages but is more prevalent in pediatric, elderly, and immune-compromised populations. RSV can infect up to 80% of children less than 1 years of age. Bronchiolitis and pneumonia are the major clinical complications in infants and young children, resulting in an estimated 51,000-82,000 hospital admissions per year in the United States. RSV infection is also an important cause of severe respiratory disease and substantial number of deaths in the elderly, with an estimated annual cost of $150 to $680 million for RSV pneumonia hospitalizations.

Given the morbidity, mortality, and economic costs associated with Influenza and RSV infections, there clearly exists a need for improved detection of these pathogens. This disclosure addresses this and other needs.

### BRIEF DESCRIPTION

This disclosure provides compositions, including kits and reagents, and methods for in vitro diagnostic analysis of Influenza A Virus (Flu A), Influenza B Virus (Flu B), Respiratory Syncytial Virus type A (RSV A) or Respiratory Syncytial Virus type B (RSV B) nucleic acids in a sample. Preferably the in vitro diagnostic analysis utilizes polymerase chain reactions (PCR), though other in vitro assay methodologies are contemplated for use with the disclosed compositions. A particularly useful in vitro assay for use with the Flu A, Flu B. RSV A or RSV B target nucleic acids is a reverse transcription PCR assay, as these target nucleic acids are RNA viruses. Conveniently, in vitro amplification assays can performed simultaneously with in vitro detection assays (real-time PCR). Thus, a particularly useful and convenient in vitro assay for use with the Flu A, Flu B, RSV A or RSV B target nucleic acids is a real-time, reverse transcription PCR assay.

It should be noted that, as used in this specification and the appended claims, the singular form “a,” “an,” and “the” include plural references unless the context clearly dictates otherwise. Thus, for example, reference to “an oligonucleotide” includes a plurality of oligonucleotides and the like. The conjunction “or” is to be interpreted in the inclusive sense, i.e., as equivalent to “and/or,” unless the inclusive sense would be unreasonable in the context.

It will be appreciated that there is an implied “about” prior to the temperatures, concentrations, times, etc. discussed in the present disclosure, such that slight and insubstantial deviations are within the scope of the present teachings herein. In general, the term “about” indicates insubstantial variation in a quantity of a component of a composition not having any significant effect on the activity or stability of the composition. All ranges are to be interpreted as encompassing the endpoints in the absence of express exclusions such as “not including the endpoints”: thus, for example, “within 10-15” includes the values 10 and 15 and all whole and partial (when applicable) values there between.

Unless specifically noted, embodiments in the specification that recite “comprising” various components are also contemplated as “consisting of” or “consisting essentially of” the recited components: embodiments in the specification that recite “consisting of” various components are also contemplated as “comprising” or “consisting essentially of” the recited components: and embodiments in the specification that recite “consisting essentially of” various components are also contemplated as “consisting of” or “comprising” the recited components (this interchangeability does not apply to the use of these terms in the claims). “Consisting essentially of” means that additional component(s), composition(s) or method step(s) that do not materially change the basic and novel characteristics of the compositions and methods described herein may be included in those compositions or methods. Such characteristics include the ability to detect a target nucleic acid present in a sample with specificity that distinguishes the target nucleic acid from other known respiratory pathogens. Any component(s), composition(s), or method step(s) that have a material effect on the basic and novel characteristics of the present disclosure would fall outside of this term.

The term “complement” refers to a nucleic acid molecule that comprises a contiguous nucleotide sequence that is complementary to a contiguous nucleic acid sequence of another nucleic acid molecule (for standard nucleotides A:T, A:U, C:G). For example 5′-AACTGUC-3′ is the complement of 5′-TTGACAG-3′. Two nucleic acid sequences are “sufficiently complementary” when, their respective contiguous nucleic acid sequences are at least 70% complementary. (see, e.g., See Sambrook, et al., Molecular Cloning, A Laboratory Manual, 2^{nd} ed. (Cold Spring Harbor Laboratory Press, Cold Spring Harbor, N.Y., 1989)).

“Perfectly matched” in reference to a nucleic acid duplex means that the poly- or oligonucleotide strands making up the duplex form a double-stranded structure, or region of double-stranded structure, with one another such that every nucleotide (or nucleotide analogue) in each strand undergoes Watson-Crick base-pairing with a nucleotide in the other strand in the duplexed (i.e., hybridized) region. The term also comprehends the pairing of nucleoside analogues, such as deoxyinosine, nucleosides with 2-aminopurine bases, and the like. Conversely, a “mismatch” in a nucleic acid duplex means that one or more pairs of nucleotides in the duplex fail to undergo Watson-Crick base-pairing.

By “substantially homologous,” “substantially corresponding”, or “substantially corresponds” is meant a nucleic acid molecule comprises a contiguous nucleic acid sequence that is at least 70% homologous to a contiguous nucleic acid sequence of another nucleic acid molecule.

A “sample” or “biological sample” is any tissue or polynucleotide-containing material obtained from a human, animal, or environmental sample and which may contain a target nucleic acid. Biological samples include peripheral blood, mucus, plasma, serum, saliva, cerebrospinal fluid, urine, or other body fluid, bone marrow, or other organ, biopsy tissue, or other materials of biological origin, as well as solutions or compositions containing materials of biological origin, for example, a bronchial lavage fluid. Samples can be obtained from a number of sources, including a clinical source wherein the sample is collected in order to determine the presence or absence of a target nucleic acid in the sample and in turn provide a patient with a diagnosis. A sample may be chemically and/or mechanically treated to disrupt tissue or cell structure, thereby releasing intracellular components into a solution.

The term “nucleotide” is defined herein to include both nucleotides and nucleosides, including deoxyribonucleotides (e.g., dATP, dCTP, dGTP, dTTP), ribonucleotides (e.g., rATP, rCTP, rGTP, rUTP), and analogues thereof. Nucleotides comprise a purine or pyrimidine base linked glycosidically to a ribose or a deoxyribose sugar and a phosphate group attached to the ribose or deoxyribose sugar. Nucleosides comprise a purine or pyrimidine base linked glycosidically to a ribose or a deoxyribose sugar, but lack the phosphate residues that are present in a nucleotide. Nucleotides and nucleosides, as used herein, refer to a monomer of DNA or RNA, respectively. (See e.g., Kornberg and Baker, DNA Replication, 2nd Ed. (Freeman, San Francisco, 1992)).

The term “analogue”, in reference to a chemical compound, refers to compound having a structure similar to that of another one, but differing from it in respect of one or more different atoms, functional groups, or substructures that are removed or replaced with one or more other atoms, functional groups, or substructures. In the context of a nucleotide or nucleoside, an analog refers to a compound that, like the nucleotide/side of which it is an analog, can be incorporated into a nucleic acid molecule (e.g., a primer, a probe and/or an amplification product). Nucleotide/side analogs are commonly added to synthetic oligonucleotides (such as primers and probes) using phosphoramidite chemistry techniques and devices. Nucleotide/side analogs are commonly added to amplification products by including the analog in a reaction mixture wherein a suitable polymerase, for example, a DNA polymerase, will incorporate the analog into the amplification product. Nucleotide/side (hereinafter “nucleotide”) analogs include synthetic nucleotides having modified base moieties and/or modified sugar moieties and/or modified phosphate groups, see, e.g., Scheit, Nucleotide Analogues (John Wiley, New York, 1980); Uhlman and Peyman, Chemical Reviews, 90:543-584 (1990), or the like. Such analogues include synthetic nucleosides designed to enhance binding properties, reduce complexity, increase specificity, and the like.

“DNA” refers to deoxyribonucleic acid, a polymer of deoxyribonucleotides linked by phosphodiester bonds. DNA can be single-stranded (ssDNA) or double-stranded (dsDNA), and can include both single and double-stranded (or “duplex”) regions. “RNA” refers to ribonucleic acid, a polymer of ribonucleotides linked by phosphodiester bonds. RNA can be single-stranded (ssRNA) or double-stranded (dsRNA), and can include both single and double-stranded (or “duplex”) regions. Single-stranded DNA (or regions thereof) and ssRNA can, if sufficiently complementary, hybridize to form double-stranded complexes (or regions). By “RNA equivalent”, “DNA equivalent”. “RNA equivalent bases” and “DNA equivalent bases” is meant RNA and DNA molecules having similar complementary base pair hybridization properties. RNA and DNA equivalents have different sugar moieties (i.e., ribose versus deoxyribose) and may differ, for example, by the presence of uracil in RNA and thymine in DNA. The differences between RNA and DNA equivalents do not contribute to differences in homology (or sequence identity) because the equivalents have the same degree of complementarity to a particular sequence.

The terms “polynucleotide” or “oligonucleotide” (used synonymously herein) mean a multimeric compound comprising two or more joined RNA nucleotides, DNA nucleotides, analogs of RNA nucleotides, analogs of DNA nucleotides, or combinations thereof. Polynucleotides can include other molecules that may be present in a joined sequence of nucleotides and that do not prevent hybridization of the polynucleotide with a second molecule having a complementary sequence. For example, a polynucleotide can include two or more joined nucleotides on a first side of a linker molecule and two or more joined nucleotides on a second side of the linker molecule, as is often the configuration of a molecular torch. Polynucleotides are preferably a polymeric chain of from 10 to 200 contiguous nucleotides. Polynucleotides may be purified from naturally occurring sources, but preferably are synthesized using any of a variety of well-known enzymatic or chemical methods. Whenever an oligonucleotide (or other nucleic acid) is represented by a sequence of letters, such as “ATGCUCTG”, unless otherwise indicated, it will be understood that the nucleotides are in 5′-3′ orientation from left to right and that “A” denotes adenosine (dATP/rATP) or an analogue thereof, “C” denotes cytidine (dCTP/rCTP) or an analogue thereof, “G” denotes guanosine (dGTP/rGTP) or an analogue thereof, “U” denotes uracil (rUTP) or an analogue thereof, and “T” denotes thymidine (dTTP) or an analogue thereof, unless otherwise noted. Usually oligonucleotides of the disclosure comprise the four natural nucleotides; however, they may also comprise non-natural nucleotide analogues.

A “probe” is an oligonucleotide that hybridizes specifically to a target nucleic acid sequence in a nucleic acid, preferably in an amplified nucleic acid, under conditions that promote hybridization, to form a detectable hybrid. Probe oligonucleotides comprise one or more of a contiguous nucleotide sequence, a target hybridizing sequence, a non-target hybridizing sequence, detectable labels, linkers, and nucleotide analogs. Probes preferably have oligonucleotide lengths from about 10 contiguous nucleotides up to 100 contiguous nucleotides. Certain specific probes that are preferred have target-hybridizing sequences in the length range of from 12-87, from 10-20, from 13-37, or from 17-23 nucleotides. A probe sequence may comprise RNA, DNA, analogs, and combinations thereof. The “backbone” of a probe may be made up of a variety of linkages known in the art, including one or more sugar-phosphodiester linkages, peptide-nucleic acid bonds (PNAs), phosphorothioate linkages, methylphosphonate linkages, or combinations thereof. Sugar moieties of the probe may be either ribose or deoxyribose, or similar compounds having known substitutions, such as, for example, 2′-O-methyl ribose and 2′ halide substitutions (e.g., 2′-O-Me or 2′-F). The nucleotide analogues incorporated into a probe oligonucleotide sequence can include inosine or “I”, 5-Me-dC, isoguanine, other derivatives of purine or pyrimidine bases, or abasic residues (e.g., nucleoside residues (e.g., The Biochemistry of the Nucleic Acids, pages 5-36, Adams, et al., ed., 11^{th} ed., 1992; PCT pub. no. WO 93/13121) The target nucleic acid sequence of a probe generally refers to a sequence contained within an amplified nucleic acid molecule that hybridizes specifically to at least a portion of the probe oligonucleotide using standard hydrogen bonding.

A probe may comprise target-specific sequences and optionally other sequences that are non-target hybridizing sequences (e.g., a sequence that does not hybridize the nucleic acid to be detected. Such non-target hybridizing sequences can include, for example, a promoter sequence, a restriction endonuclease recognition site, or sequences that contribute to three-dimensional conformation of the probe (e.g., see U.S. Pat. Nos. 5,118,801 and 5,312,728). Probes exhibiting at least some degree of self-complementarity include molecular torches and molecular beacons.

“Molecular Torches” can be designed to include distinct regions of self-complementarity (coined “the target hybridizing sequence domain” and “the target closing domain”) that are connected by a joining region and which hybridize to one another under predetermined hybridization assay conditions. When exposed to denaturing conditions, the two complementary regions (which may be fully or partially complementary) of a molecular torch melt, leaving the target hybridizing sequence domain available for hybridization to a target nucleic acid sequence when the predetermined hybridization assay conditions are restored. Molecular torches are designed so that the target hybridizing sequence domain favors hybridization to the target nucleic acid sequence over the target closing domain. The target hybridizing sequence domain and the target closing domain of a molecular torch include interacting labels (e.g., fluorescent/quencher) positioned so that a different signal is produced when the molecular torch is self-hybridized as opposed to when the molecular torch is hybridized to a target nucleic acid sequence, thereby permitting detection of probe:target duplexes in a test sample in the presence of unhybridized probe having a viable label associated therewith. Molecular Torches are described, for example, in U.S. Pat. No. 6,361,945.

A “Molecular Beacon” can be designed to have a target hybridizing sequence, an affinity pair (or nucleic acid arms) holding the probe in a closed conformation in the absence of a target nucleic acid sequence, and a label pair that interacts when the probe is in a “closed” conformation. Hybridization of the target hybridizing sequence of the molecular beacon to its intended target nucleic acid sequence separates the members of the affinity pair, thereby shifting the probe to an “open” conformation. The shift to the “open” conformation is detectable due to reduced interaction of the label pair. Molecular Beacons are described, for example, in U.S. Pat. No. 5,925,517.

A probe optionally may contain a detectable label that either may be attached to the end of the probe or attached internally on the probe. The terms “label” or “detectable label” are used interchangeably herein and refer to one or more atoms that can be specifically detected to indicate the presence of a substance to which the one or more atoms is attached. A label can be a primary label that is directly detectable or secondary label that can be indirectly detected, for example, via direct or indirect interaction with a primary. A label can be linked to polynucleotide probes either directly or indirectly. Labels include dyes, particles, chromophores (e.g., an atom or molecule that imparts a detectable color), combinatorial fluorescence energy transfer labels, electrophores, redox active moieties (e.g., transition metals), enzymes, haptens, luminescent compounds (e.g., bioluminescent, phosphorescent, or chemiluminescent moieties), fluorophores, mass labels, and radiolabels. Labels and related detections methods are well known (see e.g., U.S. Pat. No. 6,627,748 (B1); Styer and Haugland, (1967), Proc. Natl. Acad. Sci. U.S.A. 98:719; U.S. Pat. Nos. 5,591,578; 5,491,063; 5,201,015)

The term “fluorophore” means a fluorescent chemical compound that can re-emit light upon light excitation. Fluorophores include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, CalFluor Red™, CalFluor Orange™, stilbene, Quasar dyes (e.g., Quasar 570, Quasar 670, Quasar 705), Lucifer Yellow, Cascade Blue™, Texas Red, Alexa dyes, phycoerythin, Bodipy, and others known in the art, see, e.g., Haugland, Molecular Probes Handbook (Eugene, OR), 6th Edition; The Synthegen catalog (Houston, Tex.); Lakowicz, Principles of Fluorescence Spectroscopy, 2nd Ed., Plenum Press New York (1999), and WO 98/59066.

The term “quencher” is used to refer to a molecule that absorbs light. Quenchers are commonly used in combination with a light emitting label such as a fluorophore to absorb emitted light when in close proximity to the fluorophore. Quenchers are well-known in the art and include, e.g., Black Hole Quencher™ (or BHQ™, BHQ-1™, or BHQ-2™), Blackberry Quencher, Dabcyl, QSY, and Tamra™ compounds, to name a few.

A “homogeneous detectable label” refers to a label that associates with a probe oligonucleotide and that can be detected without physically removing hybridized from unhybridized forms of the label or labeled probe. Examples of homogeneous labels have been described in detail in, for example, U.S. Pat. Nos. 5,283,174; 6,150,097; 5,201,015; 5,656,207; and 5,658,737.

Linear probes, molecular torches and beacons are preferably labeled with an interactive pair of detectable labels. Examples of detectable labels that are preferred as members of an interactive pair of detectable labels interact with each other by FRET or non-FRET energy transfer mechanisms. Fluorescence resonance energy transfer (FRET) involves the radiationless transmission of energy quanta from the site of absorption to the site of its utilization in the molecule, or system of molecules, by resonance interaction between chromophores, over distances considerably greater than interatomic distances, without conversion to thermal energy, and without the donor moiety and acceptor moiety coming into kinetic collision. The “donor” is the moiety that initially absorbs and then transfers the energy, and the “acceptor” is the moiety to which the energy is subsequently transferred. In addition to FRET, there are at least three other “non-FRET” energy transfer processes by which excitation energy can be transferred from a donor to an acceptor molecule.

When the two labels of a donor/acceptor pair are held sufficiently close that energy emitted by one label can be received or absorbed by the second label, whether by a FRET or non-FRET mechanism, the two labels are said to be in “energy transfer relationship” with each other. This is the case, for example, when a molecular beacon or molecular torch is maintained in the “closed” state by formation of a stem duplex, and fluorescent emission from a fluorophore attached to one arm of the probe is quenched by a quencher moiety on the opposite arm. This is also the case when, for example, a linear probe is labeled with a fluorophore and a quencher at a distance along the linear probe that fluorescent emission from the attached fluorophore is quenched by the attached quencher. In these instances, the spatial separation of the fluorophore and quencher molecules (e.g., by “opening” the molecular torch or beacon or by hydrolyzing the linear probe molecule).

Examples of donor/acceptor pairs, include fluorescein/tetramethylrhodamine, IAEDANS/fluororescein, EDANS/DABCYL, coumarin/DABCYL, fluorescein/fluorescein, BODIPY FL/BODIPY FL, fluorescein/DABCYL, lucifer yellow/DABCYL, BODIPY/DABCYL, cosine/DABCYL, erythrosine/DABCYL, tetramethylrhodamine/DABCYL, CalOrange/BHQ1, CalRed/BHQ2, FAM/BHQ1, Quasar/BHQ2, Texas Red/DABCYL, CY5/BH1, CY5/BH2, CY3/BH1, CY3/BH2 and fluorescein/QSY7 dye. Labels are available from LGC Biosearch Technologies (Petaluma, CA), Glen Research (Sterling, VA), Integrated DNA Technologies (Skokie, Il); Thermo Fisher (Waltham, MA), and others.

Synthetic techniques and methods of bonding labels to nucleic acids and detecting labels are well known in the art (e.g., see Sambrook, et al., Molecular Cloning, A Laboratory Manual, 2^{nd} ed. (Cold Spring Harbor Laboratory Press, Cold Spring Harbor, N.Y., 1989), Chapter 10; U.S. Pat. Nos. 5,658,737, 5,656,207, 5,547,842, 5,283,174, and 4,581,333, and published European Pat. App. No. 0 747 706). A probe may optionally contain a fluorophore and a quencher. The nucleotide residues of the probe that combine with the target nucleic acid sequence need not be strictly contiguous, as may be the case with a detectable moiety internal to the sequence of the probe.

An “amplification primer” or “primer” is an optionally modified oligonucleotide that hybridizes to a target nucleic acid sequence, or its complement, and can participate in a nucleic acid amplification reaction. Primer oligonucleotides comprise one or more of a contiguous nucleotide sequence, a target hybridizing sequence, a non-target hybridizing sequence, linkers, and nucleotide analogs. Primers preferably have oligonucleotide lengths from about 10 contiguous nucleotides up to 100 contiguous nucleotides. A primer sequence may comprise RNA, DNA, analogs, and combinations thereof. The “backbone” of a primer may be made up of a variety of linkages known in the art, including one or more sugar-phosphodiester linkages, peptide-nucleic acid bonds (PNAs), phosphorothioate linkages, methylphosphonate linkages, or combinations thereof. Sugar moieties of the primer may be either ribose or deoxyribose, or similar compounds having known substitutions, such as, for example, 2′-O-methyl ribose and 2′ halide substitutions (e.g., 2′-O-Me or 2′-F). The nucleotide analogues incorporated into a primer oligonucleotide sequence can include inosine or “I”, 5-Me-dC, isoguanine, other derivatives of purine or pyrimidine bases, or abasic residues (e.g., nucleoside residues. The target nucleic acid sequence of a primer generally refers to both a sequence contained within the genetic information of an organism to be detected and a sequence contained within an amplified nucleic acid molecule that hybridizes specifically to at least a portion of the primer oligonucleotide using standard hydrogen bonding. Primers hybridize to a target nucleic acid sequence and have a 3′ end that can be extended by a DNA polymerase that incorporates nucleotides complementary to the target nucleic acid sequence to generate a double stranded portion thereof.

By “capture oligonucleotide” is meant at least one nucleic acid oligonucleotide that allows for joining of a target nucleic acid and an immobilized oligonucleotide due to base pair hybridization (preferably resulting in an immobilized probe:capture oligonucleotide:target nucleic acid complex). A capture oligonucleotide preferably includes two binding regions: a target nucleic acid-binding region and an immobilized probe-binding region, usually contiguous on the same oligonucleotide, although the capture oligonucleotide may include a target nucleic acid-binding region and an immobilized probe-binding region that are present on two different oligonucleotides joined together by one or more linkers. For example, an immobilized probe-binding region may be present on a first oligonucleotide, the target nucleic acid-binding region may be present on a second oligonucleotide, and the two different oligonucleotides are joined by hydrogen bonding with a linker that is a third oligonucleotide containing sequences that hybridize specifically to the sequences of the first and second oligonucleotides. The target hybridizing region of a capture probe can be specific for the target nucleic acid (e.g., sufficiently complementary to the target nucleic acid sequence) or non-specific for the target nucleic acid. One target capture system that includes a capture oligonucleotide is described in U.S. Pat. Nos. 6,110,678 & 9,051,601.

By “immobilized probe” or “immobilized nucleic acid” is meant a nucleic acid that joins, directly or indirectly, a capture oligonucleotide to an immobilized support. An immobilized probe is an oligonucleotide joined to a solid support that facilitates separation of bound target nucleic acids from unbound material in a sample.

The term “solid substrate” means any suitable medium present in the solid phase to which an antibody or an agent can be covalently or non-covalently affixed or immobilized.

By “separating” or “purifying” or “isolating” is meant that one or more components of the biological sample are removed from one or more other components of the sample. Sample components include nucleic acids in a generally aqueous solution phase that can also include other materials, for example, proteins, carbohydrates, lipids, and labeled probes. Preferably, the separating, isolating, or purifying step removes at least about 70%, more preferably at least about 90% and, even more preferably, at least about 95% of the other components present in the sample.

A “homogeneous assay” refers to a detection procedure that does not require physical separation of hybridized probe from non-hybridized probe prior to determining the extent of specific probe hybridization. Exemplary homogeneous assays can employ molecular beacons or other self-reporting probes that emit fluorescent signals when hybridized to an appropriate target nucleic acid sequences, chemiluminescent acridinium ester labels that can be selectively destroyed by chemical means unless present in a hybrid duplex, and other homogeneously detectable labels that will be familiar to those having an ordinary level of skill in the art.

“Amplification” refers to an in vitro procedure for obtaining multiple copies of a target nucleic acid sequence, its complement, or fragments thereof.

“Amplicon” refers to a DNA or RNA that is the product of a nucleic acid amplification or replication process. It can be formed using various methods, including polymerase chain reaction (PCR), ligase chain reaction (LCR), a transcription-associated amplification (e.g., TMA) etc.

The term “multiplex PCR” refers as a PCR reaction characterized in that two or more different amplification products, or amplicons, are generated by means of using two or more pairs of amplification primers in the same PCR reaction.

The term “multicolor” RT-PCR refers to a real time PCR assay characterized in that one or more different amplification products, or amplicons, generated either in a multiplex PCR or in a monoplex PCR (using only one pair of amplification primers) are (is) detected by using distinguishably labeled hybridization probes.

By “target nucleic acid” or “target” is meant a nucleic acid containing a target nucleic acid sequence. Described herein, target nucleic acids include Flu A nucleic acids, Flu B nucleic acids, RSV A nucleic acids and RSV B nucleic acids. By “target nucleic acid sequence”, (also referred to as “target nucleotide sequence”, “target sequence”, “target region”, “target nucleic acid molecule”), is meant a specific deoxyribonucleotide or ribonucleotide molecule or nucleotide sequence comprising all or part of the nucleotide sequence of a single-stranded nucleic acid molecule, and the deoxyribonucleotide or ribonucleotide sequence complementary thereto.

By “transcription associated amplification” is meant any type of nucleic acid amplification that uses an RNA polymerase to produce multiple RNA transcripts from a nucleic acid template. One example of a transcription associated amplification method, called “Transcription Mediated Amplification” (TMA), generally employs an RNA polymerase, a DNA polymerase, deoxyribonucleoside triphosphates, ribonucleoside triphosphates, and a promoter-template complementary oligonucleotide, and optionally may include one or more analogous oligonucleotides. Variations of TMA are well known in the art and are described, for example, in U.S. Pat. Nos. 5,437,990; 5,399,491; 5,554,516; 5,130,238; 4,868,105; and 5,124,246; published PCT application nos. WO 93/22461, WO 88/01302, WO 88/10315, WO 94/03472, and WO 95/03430.

### SUMMARY

This disclosure provides compositions, including kits and reagents, and methods for in vitro diagnostic analysis of Influenza A Virus (Flu A), Influenza B Virus (Flu B), Respiratory Syncytial Virus type A (RSV A) or Respiratory Syncytial Virus type B (RSV B) nucleic acids in a sample. Preferably the in vitro diagnostic analysis utilizes polymerase chain reactions (PCR), though other in vitro assay methodologies are contemplated for use with the disclosed compositions. A particularly useful in vitro assay for use with the Flu A, Flu B. RSV A or RSV B target nucleic acids is a reverse transcription PCR assay, as these target nucleic acids are RNA viruses. Conveniently, in vitro amplification assays can performed simultaneously with in vitro detection assays (real-time PCR). Thus, a particularly useful and convenient in vitro assay for use with the Flu A, Flu B, RSV A or RSV B target nucleic acids is a real-time, reverse transcription PCR assay.

In one aspect, the sample is a biological sample. In one aspect the biological sample is a clinical sample. In another aspect the sample is a swab sample, for example, from nasopharyngeal (NP) swab specimens obtained from a patient. In some embodiments, the compositions and methods can be used to aid in the differential diagnosis of Flu A, Flu B, and RSV A and RSV B infections. Negative results do not preclude such infection. Conversely, positive results do not rule-out bacterial infections or co-infections with other viruses. The use of additional laboratory testing and clinical presentation may also be considered in order to obtain the final diagnosis of respiratory viral infection.

One aspect provides nucleic acid molecules that are hybridization assay probes useful for detecting Flu A, Flu B, RSV A, or RSV B target nucleic acid sequences. Preferably, such probe molecule species include a probe sequence that is substantially complementary to a probe target nucleic acid sequence in the viral genome, or an amplicon generated therefrom, being targeted for detection. In preferred embodiments, the probe target nucleic acid sequence consists of about 17 to about 100 contiguous bases contained within targeted viral genome (or amplicon generated therefrom). Preferably, a probe molecule is up to about 100 nucleotide residues in length, although lengths of between about 20-60 nucleotide residues are particularly preferred.

In the context of Flu A, in some preferred embodiments the probe comprises a sequence that is preferably SEQ NAME: FA1-F. SEQ NAME: FA1-G, SEQ NAME: FA1-H. SEQ NAME: FA1-I, SEQ NAME: FA1*-J, SEQ NAME: FA1*-K. SEQ NAME: FA1-L, SEQ NAME: FA1-M, SEQ NAME: FA1-N, SEQ NAME: FA1*-O, SEQ NAME: FA1*-P, or SEQ NAME: FA1*-Q. In other embodiments, the probe sequence is preferably SEQ NAME: FA2-R. SEQ NAME: FA2-S, SEQ NAME: FA2-T, SEQ NAME: FA2*-U, or SEQ NAME: FA2*-V (SEQ ID NOS:6 to 22). In particularly preferred embodiments, two probes, one from each of the foregoing groups, are used in tandem to target two different regions of the Flu A genome or amplification products generated therefrom.

In the context of Flu B, in preferred embodiments the probe sequence is preferably SEQ NAME: FB-B, SEQ NAME: FB-B!, SEQ NAME: FB-C. SEQ NAME: FB-C!, SEQ NAME: FB-D, SEQ NAME: FB-D!, SEQ NAME: FB-E, SEQ NAME: FB-E!, SEQ NAME: FB-F. SEQ NAME: FB-F!, SEQ NAME: FB-G, SEQ NAME: FB-G!, SEQ NAME: FB-H, SEQ NAME: FB-H!, SEQ NAME: FB-I, SEQ NAME: FB-I!, SEQ NAME: FB-J, SEQ NAME: FB-J!, SEQ NAME: FB-K. SEQ NAME: FB-K!, SEQ NAME: FB-L, SEQ NAME: FB-L!, SEQ NAME: FB-M, SEQ NAME: FB-M!, SEQ NAME: FB-N, SEQ NAME: FB-N!, SEQ NAME: FB-O, SEQ NAME: FB-O!, SEQ NAME: FB-Q. SEQ NAME: FB-R. SEQ NAME: FB-S. SEQ NAME: FB-T. SEQ NAME: FB-U. SEQ NAME: FB-V. SEQ NAME: FB*-W, or SEQ NAME: FB*-X (SEQ ID NOS:30 to 57 & 59 to 66).

In the context of RSV A, in preferred embodiments the probe sequence is preferably SEQ NAME: RA-A, SEQ NAME: RA-E, SEQ NAME: RA-F. SEQ NAME: RA-G, SEQ NAME: RA-H, SEQ NAME: RA-J, SEQ NAME: RA-J!, SEQ NAME: RA-K, SEQ NAME: RA-K!, SEQ NAME: RA-L, SEQ NAME: RA-L!, SEQ NAME: RA-M, SEQ NAME: RA-M!, SEQ NAME: RA-O, SEQ NAME: RA-P. SEQ NAME: RA-Q. SEQ NAME: RA*-W, and SEQ NAME: RA*-X (SEQ ID NOS:71, 75 to 78, 80 to 87, 89 to 91, 97 & 98).

In the context of RSV B, in preferred embodiments the probe sequence is preferably SEQ NAME: RB-D. SEQ NAME: RB-E, SEQ NAME: RB-V. SEQ NAME: RB-V!, SEQ NAME: RB-W. SEQ NAME: RB-W!, SEQ NAME: RB-X, SEQ NAME: RB-X!, SEQ NAME: RB-Y, and SEQ NAME: RB-Y! (SEQ ID NOS: 102, 103, & 107 to 114).

Preferably, a probe molecule species is labeled, optionally distinguishably labeled such that any one probe molecule species can be distinguished from other probe molecule species in a multiplex detection assay. Distinguishable labeling can be achieved using two or more detectable labels, for example, a chemiluminescent moiety, a fluorophore moiety, and both a fluorophore moiety and a quencher moiety.

Another aspect the disclosure concerns nucleic acid molecules that are amplification primers engineered for use in in vitro amplification of target nucleic acid sequences. A related aspect of the disclosure relates to pairs of such primers that can be used to amplify desired amplicons that contain a target nucleic acid sequence. These primers include one or more of the following primers pairs: a first Flu A primer pair, a second Flu A primer pair that can be used to amplify a region of the Flu A target nucleic acid that is different from the region of the Flu A target nucleic acid that can be amplified using the first Flu A primer pair, a Flu B primer pair, an RSV A primer pair, and an RSV B primer pair. These primer pairs include first and second primers that can be used generate corresponding amplicons for Flu A, Flu B. RSV A, and/or RSV B if the viral pathogen is present in the biological sample being tested.

In general, a primer pair includes a first primer that includes a priming nucleotide sequence that is substantially complementary to a first target nucleic acid sequence of viral genome a portion of which is to be amplified. Preferably, the first and second target nucleic acid sequences are spaced apart in the target nucleic acid by at least 10, and preferably by about 50-1,000 nucleotides, and each of them preferably consists of about 17 to about 100 contiguous bases of the viral genome to be detected. In some embodiments, one or more of the primers in one or more primer pairs further comprises a primer upstream region having a nucleotide sequence that is not complementary to the primer's target nucleotide sequence.

Preferred first primers for generating a first Flu A amplicon have the priming nucleotide sequence of SEQ NAME: FA1-A or SEQ NAME: FA1-W. Preferred second primers useful with such first primers have the priming nucleotide sequence of SEQ NAME: FA1-Y or SEQ NAME: FA1-AB. SEQ ID NOS:1, 23, 25, & 28.

Preferred first primers for generating a second Flu A amplicon have the priming nucleotide sequence of SEQ NAME: FA2-B, SEQ NAME: FA2-C, SEQ NAME: FA2-D, SEQ NAME: FA2-E, or SEQ NAME: FA2-X. Preferred second primers useful with such first primers have the priming nucleotide sequence of SEQ NAME: FA2-Z or SEQ NAME: FA2-AA. SEQ ID NOS:2, 5, 24, 26, & 27.

Preferred first primers for generating a Flu B amplicon have the priming nucleotide sequence of SEQ NAME: FB-A or SEQ NAME: FB-Y. Preferred second primers useful with such first primers have the priming nucleotide sequence of SEQ NAME: FB-Z, SEQ NAME: FB-AA, and SEQ NAME: FB-AB. SEQ ID NOS:29 & 67 to 70.

Preferred first primers for generating an RSV A amplicon have the priming nucleotide sequence of SEQ NAME: RA-I or SEQ NAME: RA-N. Preferred second primers useful with such first primers have the priming nucleotide sequence of SEQ NAME: RA-B, SEQ NAME: RA-C, SEQ NAME: RA-D, SEQ NAME: RA-R, SEQ NAME: RA-S, SEQ NAME: RA-T, SEQ NAME: RA-U, and SEQ NAME: RA-V. SEQ ID NOS:79, 88, 72 to 74, & 92 to 96.

Preferred first primers for generating an RSV B amplicon have the priming nucleotide sequence of SEQ NAME: RB-A, SEQ NAME: RB-B, SEQ NAME: RB-C, and SEQ NAME: RB-U. Preferred second primers useful with such first primers have the priming nucleotide sequence of SEQ NAME: RB-F, SEQ NAME: RB-G, SEQ NAME: RB-U, and SEQ NAME: RB-Z. SEQ ID NOS:99 to 101, 104 to 106, & 115.

In some preferred embodiments, a probe and/or a primer contains one or more methylated cytosine bases.

Another related aspect of the disclosure concerns compositions that contain such probes, primers, and primer pairs. Such compositions include dry or liquid compositions. Dried compositions include lyophilized reagents containing one or more of a primer and a probe.

Another aspect of the disclosure relates to kits that include primers and/or probes. Such kits can also include salts, enzymes, dNTPs, dRTPs, other substrates, and/or instructions for use of such materials. The primers, probes, salts, enzymes, dNTPs, rNTPs, and/or other substrates of the kit may be in a dried form or in an aqueous form.

Another aspect of the disclosure relates to a reagent that contains primers and/or probes. Such reagents can also include salts, enzymes, dNTPs, rNTPs, and/or other substrates. The primers, probes, salts, enzymes, dNTPs, rNTPs, and/or other substrates of the reagents may be in a dried form or in an aqueous form.

Still another aspect of the disclosure concerns methods of using such primers and probes to analyze samples to determine if the sample contains one or more of a Flu A target nucleic acid, Flu B target nucleic acid, RSV A target nucleic acid, and RSV B target nucleic acid. The foregoing and other objects, features, and advantages of the compositions and methods will be apparent from the following detailed description and the claims.

### DETAILED DESCRIPTION

Described herein are compositions, including kits and reagents, and methods for selectively detecting nucleic acids of various viral pathogens, specifically, Influenza A (Flu A), Influenza B (Flu B), Respiratory Syncytial Virus A (RSV A), and Respiratory Syncytial Virus B (RSV B), in a sample. These compositions and methods can be used, for example, in diagnostic applications, for screening clinical samples, nasopharyngeal samples, bronchoalveolar samples, donated blood and blood products or other tissues that may contain one or more of these pathogenic organisms.

As will be appreciated, any primer and probe sequences specific for Flu A, Flu B, RSV A, RSV B and/or other pathogenic viral target may be used as primers or probes in any suitable primer/probe-based in vitro nucleic acid amplification method adapted for amplification of an intended target nucleic acid. It is also understood that oligonucleotides having the sequences described herein could serve alternative functions in assays for detecting viral target nucleic acids. For example, a probe could be used as a primer (e.g., as one member of primer pair), and a primer could be used as a probe in an alternative detection assay.

The amplification primers are useful as components of uniplex or multiplex amplification reactions wherein amplicon species can be produced from target-specific primers in the reaction mixture. A multiplex amplification reaction includes primer pairs for amplifying two or more of Flu A, Flu B, RSV A, and RSV B, or, additionally includes primers for one or more of Flu A, Flu B, RSV A, and RSV B and one or more additional targets (e.g., human metapneumovirus, rhinovirus, adenovirus, parainfluenza virus, and/or bordetella).

Amplification methods useful in connection with the present disclosure include: Polymerase Chain Reaction (PCR); Transcription-Mediated Amplification (TMA); Nucleic Acid Sequence-Based Amplification (NASBA); Strand Displacement Amplification (SDA); and amplification methods using self-replicating polynucleotide molecules and replication enzymes such as MDV-1 RNA and Q-beta enzyme. Methods for carrying out these various amplification techniques respectively can be found in U.S. Pat. Nos. 4,965,188; 5,399,491; 5,455,166; and 5,472.840, published European patent application EP 0 525 882, and Lizardi, et al., BioTechnology 6:1197 (1988). In particularly preferred embodiments, Flu A, Flu B, RSV A, and RSV B nucleic acid sequences are amplified using real-time PCR (RT-PCR).

Due to the lack of sequence conservation among respiratory virus strains, particularly for Flu A, and to accommodate for mismatches/mutations between a primer or a probe and their corresponding target nucleic acid sequences in viral target nucleic acid, degenerate bases and non-Watson Crick (NWC) base pairing can, in some preferred embodiments, be included in a primer or probe oligonucleotide. A NWC position in an oligonucleotide refers to a position where the oligonucleotide is configured to hybridize to at least one target nucleic acid sequence with a non-Watson Crick pairing, such as G-U, G-T, or G-A (either the G or the U/T/A can be the base in the oligonucleotide). In some embodiments, the NWC position is configured to hybridize via a wobble (G-U or G-T) or purine-purine (G-A) pair. In some embodiments, when one or more degenerate bases have been identified in the target nucleic acid sequence for a single primer or probe, multiple primer species or probe species may be synthesized in order to include all base combinations.

Useful guidelines for designing amplification primers and probes with desired characteristics are known in the art, and are described herein. The optimal sites for amplifying and probing Flu A, Flu B. RSV A, and RSV B nucleic acids contain two, and preferably three, conserved regions each greater than about 15 bases in length, all spatially separated from one another within a region of about 1.000, preferably of about 500, and even more preferably, of about 200 bases of contiguous sequence of the target nucleic acid. The degree of amplification observed with a set of primers depends on several factors, including the ability of the primers to hybridize to their complementary sequences and their ability to be extended enzymatically. Because the extent and specificity of hybridization reactions are affected by a number of factors, manipulation of those factors will determine the exact sensitivity and specificity of a particular oligonucleotide, whether perfectly complementary to its target or not. The effects of varying assay conditions are known in the art, see, e.g., U.S. Pat. No. 5,840,488.

Amplification primers and probes should be positioned to minimize the stability of oligonucleotide:nontarget (e.g., nucleic acid with similar sequence to target nucleic acid) and oligonucleotide:oligonucleotide (e.g., primer dimers and self-complementarity) nucleic acid hybrids. It is preferred that the amplification primers and detection probes be able to distinguish between target and non-target sequences. In designing primers and probes, the differences in their melting temperature (Tm) values for oligonucleotide:target compared to oligonucleotide:non-target and oligonucleotide:oligonucleotide should be as large enough to favor oligonucleotide:target hybridizaztion. Also, long homopolymer tracts and high GC content are preferably avoided to reduce spurious primer extension.

As is known, nucleic acid hybridization involves the association of two single strands of complementary nucleic acid to form a hydrogen-bonded double strand. It is implicit that if one of the two strands is wholly or partially involved in a hybrid, then that strand will be less able to participate in formation of a new hybrid. By designing primers and probes so that substantial portions of the sequences of interest are single-stranded, the rate and extent of hybridization may be greatly increased. If the target is in a double-stranded form (as is the case with PCR products), denaturation prior to hybridization will typically be required.

Primers useful for conducting amplification reactions can have different lengths to accommodate the presence of extraneous sequences that do not participate in target binding, and that may not substantially affect amplification or detection procedures. For example, promoter-primers useful for performing amplification reactions in accordance with the disclosure have at least a minimal sequence that hybridizes to the desired target nucleic acid sequence, and a promoter sequence positioned upstream of that minimal sequence. However, insertion of sequences between the target binding sequence and the promoter sequence could change the length of the primer without compromising its utility in the amplification reaction. Additionally, the lengths of the amplification primers and detection probes are matters of choice as long as the sequences of these oligonucleotides conform to the minimal essential requirements for hybridizing with the desired complementary target sequence.

Hybridization assay probes useful for detecting Flu A, Flu B, RSV A, and RSV B nucleic acid sequences include a sequence of bases substantially complementary to the selected target nucleic acid sequence in the Flu A. Flu B. RSV A. or RSV B genome (or amplicon representing the corresponding region and its flanking or surrounding regions). Such probes may optionally have additional bases outside of the targeted nucleic acid region. which may or may not be complementary to Flu A. Flu B, RSV A, or RSV B nucleic acid.

Preferred probes are sufficiently homologous to the target nucleic acid to hybridize under stringent hybridization conditions corresponding to a designed amplification and detection reaction. For example, in PCR they extension and detection reactions are carried out such that an oligonucleotide would hybridize to its target nucleic acid sequence at a reaction temperature of about 60º C. Salt concentrations also impact hybridization of an oligonucleotide to its target nucleic acid sequence. An exemplary salt concentration is in a suitable range of about 0.6-0.9 M. Preferred salts include lithium chloride, but other salts such as sodium chloride and sodium citrate also can be used in the hybridization solution. Example high stringency hybridization conditions are also provided by 0.48 M sodium phosphate buffer, 0.1% sodium dodecyl sulfate, and 1 mM each of EDTA and EGTA, or by 0.6 M LiCl, 1% lithium lauryl sulfate, 60 mM lithium succinate and 10 mM each of EDTA and EGTA. Those skilled in the art are familiar with preparing solutions for nucleic acid hybridizations.

Probes in accordance with the disclosure have sequences complementary to, or corresponding to, a pre-selected target region of particular viral target nucleic acid targeted by the probe. Preferred probes have a probe sequence, which includes the target-hybridizing sequence of bases together with any base sequences that are not complementary to the nucleic acid that is to be detected, in the length range of from 10-100 nucleotides.

Amplification of nucleic acids by polymerase chain reaction (PCR) is a fundamental technique in molecular biology, typically requiring sample preparation, amplification, and product analysis. Although these steps are usually performed sequentially, amplification and analysis can occur simultaneously. DNA dyes or fluorescent probes can be added to the PCR mixture before amplification and used to analyze PCR products during amplification. Sample analysis occurs concurrently with amplification in the same tube within the same instrument. Such a combined approach decreases sample handling, saves time, and greatly reduces the risk of product contamination for subsequent reactions, as there is no need to remove the samples from their closed containers for further analysis. The concept of combining amplification with product analysis has become known as “real time” PCR (RT-PCR). See, for example, U.S. Pat. Nos. 6,174,670 and 8,137,616. In real time PCR, the formation of PCR products is monitored in each cycle of the PCR. The amplification is usually measured in thermocyclers that have additional devices for signal generation and detection from labels attached to probe oligonucleotide species during the amplification reaction. A number of such devices are known in the art for performing multiplex diagnostic assays with three, four, or more distinguishably labeled hybridization probes within one reaction vessel.

As is known, different formats exist for probe-based, real time detection of amplified DNA in multiplex assays. Common examples include “Taqman” probe systems, Molecular beacons and torches, single labeled probes, and FRET hybridization probes.

In TaqMan probe formats, a single-stranded hybridization probe for a given target is labeled with a donor/acceptor pair of detectable labels. When the donor (e.g., a fluorophore moiety) is excited with light of a suitable wavelength, the absorbed energy is transferred to the acceptor, (e.g., a quencher moiety), according to the principle of FRET. During the annealing step of a PCR reaction cycle, the hybridization probe binds to the target DNA and is degraded by the 5′-3′ exonuclease activity of the Taq polymerase during the subsequent elongation phase. As a result, the excited donor moiety and the acceptor moiety become spatially separated, thus allowing for unquenched signal from the donor (e.g., a fluorescent emission) that is detected by the device. See, e.g., U.S. Pat. No. 5,538,848.

Molecular beacon and torch formats typically also include hybridization probes labeled with a donor/acceptor pair, with each of the donor moiety and the acceptor moiety being located at opposite ends of the probe. As a result of the secondary structure of the probe, which often involves hybridization of complementary regions at the ends of the probe, both the donor moiety and the acceptor moiety (e.g., the fluorescent moiety and the quencher moiety) are in spatial vicinity in solution. After hybridization of the probe's target hybridizing region to the desired target nucleic acid sequence, the donor moiety and the acceptor moiety are separated from one another such that after excitation of the donor moiety with light of a suitable wavelength its emission can be measured. See, e.g., U.S. Pat. No. 5,118,801.

In single label probe (SLP) formats, a single oligonucleotide is labeled with a single fluorescent dye at either the 5′- or 3′-end. Different designs can be used for oligonucleotide labeling, such as G-Quenching probes and Nitroindole-Dequenching probes. In G-Quenching embodiments, the fluorescent dye is attached via a C at the oligonucleotide's 5′- or 3′-end. Fluorescence decreases significantly when the probe is hybridized to the target if two G's are located on the target strand opposite to C and in position 1 aside of the complementary oligonucleotide probe. In the Nitroindole Dequenching embodiments, the fluorescent dye is attached to nitroindole at the 5′- or 3′-end of the oligonucleotide, and the itroindole decreases the fluorescent signaling from free (e.g., unhybridized) probe molecules. Fluorescence increases when the probe hybridizes to the target DNA due to a dequenching effect.

Multiplex assays that use FRET hybridization probes to detect target nucleic acids are particularly useful in homogenous hybridization assays (see, e.g., Matthews and Kricka, Analytical Biochemistry, vol. 169 (1988), pp: 1-25). In particular, the FRET hybridization probe format can be used in RT-PCR to detect amplified target DNA species.

Besides PCR and real time PCR, FRET hybridization probes can also be used for melting curve analysis. In such an assay, the target nucleic acid is amplified first in a typical PCR reaction with suitable amplification primers. The hybridization probes may already be present during the amplification reaction or added subsequently. After completion of the PCR reaction, the temperature of the sample is steadily increased, and fluorescence is detected as long as the hybridization probe is bound to the target DNA. At the melting temperature, the hybridization probe molecules are released from their complementary target sequences, and the fluorescent signal decreases immediately to the background level. This decrease is monitored with an appropriate fluorescence versus temperature-time plot such that a first derivative value can be determined, at which the maximum of fluorescence decrease is observed.

In some preferred embodiments, RT-PCR methods for amplifying and detecting multiple target DNA sequences in a multiplex assay are used. Such methods involve providing a composition or reaction mixture containing nucleic acids from biological sample, probes, primers, and a suitable polymerase activity to catalyze amplification, subjecting the reaction mixture to a thermocyling protocol such that amplification of the multiple target sequences occurs, and monitoring hybridization of each of the probe molecule species (e.g., pairs of FRET hybridization probes) at least once after a plurality of amplification cycles. In embodiments where the viral target nucleic acid(s) to be detected is/are comprised of one or more RNA molecules, such methods typically involve first converting RNA to DNA (e.g., a “complementary” DNA or “cDNA”) through the use of a reverse polymerase activity.

In such multiplex embodiments, the composition or reaction mixture typically comprises at least 2, preferably 3-5, and most preferably 4 pairs of detection probes, preferably each pair of probes comprising a FRET donor moiety and a FRET acceptor moiety. In addition, such a composition or reaction mixture also comprises a number of reagents, including one or more of the following: buffers designed for PCR, dNTPs, a template dependent DNA polymerase (preferably a thermostable DNA polymerase), a reverse transcriptase.

During or after the amplification process is complete, the reaction is monitored to detect stable hybridization between one or more of the distinguishably labeled probe species present in the reaction and its corresponding target nucleic acid sequence (carried in an amplicon generated using the corresponding primer pair for the particular viral (or other) pathogen to be detected. Based on whether the donor moieties from each of the different donor/acceptor pairs are detected, it can then be determining if the biological sample contains Flu A, Flu B. RSV A, and/or RSV B and/or such other pathogens as are targeted in the particular assay.

Certain preferred kits will comprise one or more of a probe, a primer, a capture oligonucleotide, internal control oligonucleotides other ancillary oligonucleotides a buffer, dNTPs. DNA polymerase, reverse transcriptase, and instructions for using components of the kit (or a link to a website providing such instructions).

The following examples are provided to illustrate certain disclosed embodiments and are not to be construed as limiting the scope of this disclosure in any way.

General Reagents and Methods. Unless otherwise indicated, amplifications were performed using an ABI 7500 FAST® instrument. Viral isolates used as amplification targets or controls were diluted in suitable media, e.g., Micro Test M4 media (Remel Inc. Cat. No. R12500), Micro Test M5 Viral Transport Medium (Remel, Inc. Cat. No. R12515), Micro Test M6 Viral Transport Medium (Remel, Inc. Cat. No. R12530), Micro Test M4RT Viral Transport Medium (Remel, Inc. Cat. No. R12505), or Copan Universal Transport Medium (Copan Diagnostics, Inc., Cat. No. 330C). Nucleic acid was extracted from viral isolates using a non-specific target capture procedure as described in US Patent App. Pub. 2013/0209992.

PCR reaction mixtures were typically assembled as follows: 19.05 uL Supermix (Promega GoTaq® Supermix); 0.35 uL MMLV Reverse Transcriptase (35 U); 0.6 uL GoTaq MDX Hotstart Taq (3U); 5 uL of nucleic acids (primers, probe, and target in suitable diluent);=25 uL total reaction volume. Promega, Madison, WI; New England Biolabs, Ipswich, MA; Sigma-Aldrich, St. Louis MO; Thermo Fisher, Waltham, MA; and others.

### Example 1

### Multiplex RT-PCT Assay to Detect Flu A, Flu B, RSV A, and RSV B

This example describes a representative RT-PCR assay based on Taqman reagent chemistry to provide for the detection and differentiation of Influenza A Virus, Influenza B Virus, and Respiratory Syncytial Virus Types A and B in a biological sample.

Here, the process begins by collecting, for example, a nasopharyngeal swab specimen from a symptomatic human patient. Unless the sample is to be immediately assayed, the sample is preferably placed in sealable container (e.g., an RNase/DNase-free 1.5 mL polypropylene microcentrifuge tube) along with an appropriate volume of viral transport medium (VTM; e.g., Remel, Inc., Copan Diagnostics, Inc., or (Becton, Dickinson and Co.). Preferably, a Universal Internal Control (UIC) is also then added to the sample to monitor for inhibitors that may be present in the sample.

Next, nucleic acids in the sample are isolated, for example, by using a MagNA Pure LC System (Roche) and a MagNA Pure Total Nucleic Acid Isolation Kit (Roche; cat. no. 03038505001) or a NucliSENS easyMAG System (bioMérieux) and an Automated Magnetic Extraction Reagents (bioMérieux). Purified nucleic acids are then added to a reaction mix along with a thermostable DNA polymerase and a reverse transcriptase. The reaction mix contains oligonucleotide primer pairs and target-specific oligonucleotide probes for each of Flu A. Flu B, RSV A, and RSV B, as well as Taq DNA polymerase, buffer containing dNTPs (dATP, dCTP, dGTP, dTTP (or dUTP)), MgCl₂, and stabilizers, and bovine serum albumin. For reverse transcription of viral genomes, M-MLV Reverse Transcriptase can be used, and to protect RNA from degradation, an RNase inhibitor (e.g., RNase Inhibitor II) can also be included. Various control nucleic acids may also be included. Such controls may be, for example, non-infectious in vitro transcribed RNA of specific viral sequences and/or non-infectious plasmid DNA containing control sequences. If desired, two different sets of amplification primers and probes targeting different genomic regions of the viruses to be detected can be used for any given target genome, particularly when, as may be the case with Flu A, genetic variation between strains may be such that detection based on a single region may be insufficient to assure accurate analysis. The amplification primers of the various primer pairs are complementary to highly conserved regions of genetic sequences for these respiratory viruses. The probe species are each dual-labeled with a distinguishable reporter dye and a quencher.

Reverse transcription of RNA into cDNA and subsequent amplification of DNA may be performed, for example, on a Cepheid SmartCycler II instrument (Cepheid, Sunnyvale, CA). In this process, for each viral genome to be detected, the probe species for the target viral genome (or region thereof) anneals specifically to the target nucleotide sequence of the target nucleic acid molecule (e.g., a specific region of the Flu A genome), followed by primer extension and amplification. The Taqman reagent chemistry utilizes the 5′-3′ exonuclease activity of the Taq polymerase to cleave the probe, thus separating the reporter dye from its quencher. This generates an increase in fluorescent signal upon excitation from a light source. With each cycle, additional reporter dye molecules are cleaved from their respective probes, further increasing the fluorescent signal. The amount of fluorescence at any given cycle is dependent on the number of amplification products (amplicons) present at that time. Fluorescence intensity is monitored during each PCR cycle by the real-time instrument.

### Example 2

### Amplification and Detection of Flu A, Flu B, RSV A & RSV B in Clinical Samples

Remnant nasopharyngeal (NP) swab and lower and lower respiratory tract (LRT) specimens from individuals exhibiting signs and/or symptoms of a respiratory tract infection were analyzed in a multiplex real-time PCR assay using primers and probes for the amplification and detection of Flu A, Flu B, RSV A and RSV B target nucleic acids. NP swab and LRT samples were tested with the Panther Fusion Flu A/B/RSV assay.

For this example, 2930 remnant NP swab specimen were used. The specimen were processed to release nucleic acids. Briefly, remnant NP swab specimen were received in Remel transport media (Thermo Fisher, Waltham, MA). An aliquot of the transport media (500 ul) from each specimen was separately combined with a lysis reagent (710 ul) in a Panther Fusion Lysis Tube (Hologic, Marlborough, MA). Following an incubation, 360 ul of lysed specimen was combined with 450 ul of a target nucleic acid isolation reagent containing a capture oligonucleotide and a solid support. The target nucleic acid isolation reaction was performed on a Panther Fusion device (Hologic, Marlborough, MA), and as generally described in U.S. Pat. Nos. 6,110,678 & 9,051,601. Target nucleic acids isolated from each clinical specimen were then eluted from the capture reaction into a 50 ul eluate to provide 2930 sample conditions, each corresponding to one of the NP swab specimen. A nucleic acid amplification and detection reaction was set-up as follows: 5 ul from each sample condition was added to a well of a multiwall plate. Also contained within the well was 20 ul of a rehydrated real-time PCR reaction mixture. The dried PCR reaction mixture was rehydrated using 24 ul of a magnesium salt containing buffer. Components of this real-time PCR reaction mixture are described above and further comprised primers and probes with nucleotide sequences illustrated as SEQ ID NOS:5, 7, 12, 18, 23, 25 to 27, 64, 67, 68, 75, 79, 92, 101, 102, & 115. Probes for detecting Flu A amplification products were labeled with FAM/BHQ1, probes for detecting Flu B amplification products were labeled with CalRed/BHQ2, and probes for detecting RSV A and RSV B amplification products were labeled with CalOrange/BHQ1 (labels available from LGC Biosearch Technologies, Petaluma, CA). Each sample condition was independently added to a PCR reaction microtube. Control wells included an internal control, a positive control and a negative control.

Each PCR reaction microtube was then placed on a Panther Fusion device (Hologic, Inc., Marlborough, MA) and analyzed for the presence or absence of one or more of the target nucleic acids in each well. Of the 2930 NP swab specimen, 61 provided inconsistent results, and thus were deemed invalid and excluded from the evaluation results; 189/2869 (6.6%) were positive for Flu A target nucleic acid; 55/2869 (1.9%) were positive for Flu B target nucleic acid; and 365/2869 (12.7%) were positive for RSV A and/or RSV B.

A similar assay was performed using the remnant lower respiratory tract (LRT) specimen, with the exception that 250 ul of the LRT specimen was combined with 250 ul of lysis reagent, and then 360 ul of this combined solution was used for the target nucleic acid reaction. For this example, 144 remnant LRT specimen were used. The specimen were treated (specimen lysis, nucleic acid isolation, amplification, and detection) as is generally described above in this example. Of the 144 LRT specimen, 4 provided inconsistent results or were not tested, and thus were deemed invalid and excluded from the evaluation results; 3/140 (2.1%) were positive for Flu A target nucleic acid; 0/140 (0.0%) were positive for Flu B target nucleic acid; and 1/140 (0.7%) were positive for RSV A and/or RSV B.

These results show that the assay is a sensitive and specific assay for the detection of target nucleic acids from NP swab specimen. These results also show that the assay is sensitive for the detection of Flu A target nucleic acids, but on these LRT specimen sensitivity could not be determined for Flu B and RSV A & B target nucleic acids. These results show that the assay has high specificity for Flu A, Flu B, RSV A and RSV B target nucleic acids.

### Example 3

### Exemplary Oligonucleotide Sequences

Table 1 illustrates a number of primer and probe sequences that are useful as compositions, in kits, as diagnostic reagents, and/or in methods for the amplification or detection of one or more of Flu A, Flu B, RSV-A, and RSV-B. The following Table 1 illustrates only the nucleotide sequences. It is understood that these sequences may further include detectable labels, sugar modifications (e.g., 2′-methoxy), base modifications (e.g., a methylated base), and other chemical components that are not represented in the illustrated contiguous arrangements of symbols.

TABLE 1

| | | | Oligo- |
|---|---|---|---|
| | SEQ | | nucleotide |
| SEQ ID NO: | Name: | Sequence* | Type |
| SEQ ID NO: 1 | FA1-A | GATCTTGAGGCTCTCATG | Primer |
| SEQ ID NO: 2 | FA2-B | ATAACRTTCCATGGRGCCAA | Primer |
| SEQ ID NO: 3 | FA2-C | ATAACRTTCCATGGGGCCAA | Primer |
| SEQ ID NO: 4 | FA2-D | ATAACGTTCCATGGRGCCA | Primer |
| SEQ ID NO: 5 | FA2-E | ATAACGTTCCATGGGGCCAA | Primer |
| SEQ ID NO: 6 | FA1-F | CCCTTAGTCAGAGGTGACAG | Probe |
| SEQ ID NO: 7 | FA1-G | TCAGGCCCCCTCAAAGCCGAGATCGCC | Probe |
| SEQ ID NO: 8 | FA1-H | TCAGGCCCCCTCAAAGCCGARATCGC | Probe |
| SEQ ID NO: 9 | FA1-I | TCAGGCCCCCTCAAAGCCGAGATCGC | Probe |
| SEQ ID NO: 10 | FA1*-J | 3′-AGUCCGGGGGAGUUUCGGCTUUAGCG-5′ | Probe |
| SEQ ID NO: 11 | FA1*-K | 3′-AGUCCGGGGGAGUUUCGGCTCUAGCG-5′ | Probe |
| SEQ ID NO: 12 | FA1-L | AGCCAUTCCATGAGAGCCTCAAGATCC | Probe |
| SEQ ID NO: 13 | FA1-M | AGCCAYTCCATRAGAGCCTCAAGATC | Probe |
| SEQ ID NO: 14 | FA1-N | AGCCAUTCCATGAGAGCCTCAAGATC | Probe |
| SEQ ID NO: 15 | FA1*-0 | 3′-UCGGUGAGGUACUCUCGGAGUUCUAG-5′ | Probe |
| SEQ ID NO: 16 | FA1*-P | 3′-UCGGUAAGGUACUCUCGGAGUUCUAG-5′ | Probe |
| SEQ ID NO: 17 | FA1*-Q | 3′-UCGGUAAGGUAUUCUCGGAGUUCUAG-5′ | Probe |
| SEQ ID NO: 18 | FA2-R | CTGGTGCACTTGCCAGTTGUATGC | Probe |
| SEQ ID NO: 19 | FA2-S | CTGGTGCACTTGCCAGTTCYATG | Probe |
| SEQ ID NO: 20 | FA2-T | CTGGTGCACTTGCCAGTTCUATG | Probe |
| SEQ ID NO: 21 | FA2*-U | 3′-GACCACGUGAACGGUCAAGAUAC-5′ | Probe |
| SEQ ID NO: 22 | FA2*-V | 3′-GACCACGUGAACGGUCAAGGUAC-5′ | Probe |
| SEQ ID NO: 23 | FA1-W | CTTCTAACCGAGGTCGAAACGT | Primer |
| SEQ ID NO: 24 | FA2-X | ATAACGTTCCATGGGGCCAA | Primer |
| SEQ ID NO: 25 | FA1-Y | CCCTTAGTCAGAGGTGACA | Primer |
| SEQ ID NO: 26 | FA2-Z | CCCATTCTGTTGTATATGAG | Primer |
| SEQ ID NO: 27 | FA2-AA | CCCATCCTGTTGTATATGAG | Primer |
| SEQ ID NO: 28 | FA1-AB | GGTGAGCGTGAACACAAA | Primer |
| SEQ ID NO: 29 | FB-A | AGTGGAGGATGAAGAAGATGGC | Primer |
| SEQ ID NO: 30 | FB-B! | GCCTGCTTTGCCTTCTCCATCTTCTGTGCAGGC | Torch |
| SEQ ID NO: 31 | FB-B | GCCTGCTTTGCCTTCTCCATCTTCTG | Probe |
| SEQ ID NO: 32 | FB-C! | GCCTGCTTTGCCTTCTCCATCTTCTGTAGCAGGC | Torch |
| SEQ ID NO: 33 | FB-C | GCCTGCTTTGCCTTCTCCATCTTCTGT | Probe |
| SEQ ID NO: 34 | FB-D! | GCGCTAGTTCTGCTTTGCCTTCTCCATCTTCCTAGCGC | Torch |
| SEQ ID NO: 35 | FB-D | GCGCTAGTTCTGCTTTGCCTTCTCCATCTTC | Probe |
| SEQ ID NO: 36 | FB-E! | GCGCTAGTTCTGCTTTGCCTTCTCCATCTTCCTAGCGC | Torch |
| SEQ ID NO: 37 | FB-E | GCGCTAGTTCTGCTTTGCCTTCTCCATCTTC | Probe |
| SEQ ID NO: 38 | FB-F! | GCGCTAGTTCTGCTTTGCCTTCTCCATCTTCCTAGCGC | Torch |
| SEQ ID NO: 39 | FB-F | GCGCTAGTTCTGCTTTGCCTTCTCCATCTTC | Probe |
| SEQ ID NO: 40 | FB-G! | GCTGCTAGTTCTGCTTTGCCTTCTCCATCGCAGC | Torch |
| SEQ ID NO: 41 | FB-G | GCTGCTAGTTCTGCTTTGCCTTCTCCATC | Probe |
| SEQ ID NO: 42 | FB-H! | GCCTGCTAGTTCTGCTTTGCCTTCTCCATCGCAGGC | Torch |
| SEQ ID NO: 43 | FB-H | GCCTGCTAGTTCTGCTTTGCCTTCTCCATC | Probe |
| SEQ ID NO: 44 | FB-I! | GCCTGCTAGTTCTGCTTTGCCTTCTCCATCGCAGGC | Torch |
| SEQ ID NO: 45 | FB-I | GCCTGCTAGTTCTGCTTTGCCTTCTCCATC | Probe |
| SEQ ID NO: 46 | FB-J! | GCCTGCTAGTTCTGCTTTGCCTTCTCCATCAGCAGGC | Torch |
| SEQ ID NO: 47 | FB-J | GCCTGCTAGTTCTGCTTTGCCTTCTCCATC | Probe |
| SEQ ID NO: 48 | FB-K! | GCCTGCTAGTTCTGCTTTGCCTTCTCCATCAGCAGGC | Torch |
| SEQ ID NO: 49 | FB-K | GCCTGCTAGTTCTGCTTTGCCTTCTCCATC | Probe |
| SEQ ID NO: 50 | FB-L! | GCCTGCTAGTTCTGCTTTGCCTTCTCCATCAGCAGGC | Torch |
| SEQ ID NO: 51 | FB-L | GCCTGCTAGTTCTGCTTTGCCTTCTCCATC | Probe |
| SEQ ID NO: 52 | FB-M! | GCCTGCTAGTTCTGCTTTGCCTTCTCCATCTAGCAGGC | Torch |
| SEQ ID NO: 53 | FB-M | GCCTGCTAGTTCTGCTTTGCCTTCTCCATC | Probe |
| SEQ ID NO: 54 | FB-N! | GCGGAGAAGGCAAAGCAGAAUTAGCAGTCTCCGC | Torch |
| SEQ ID NO: 55 | FB-N | GCGGAGAAGGCAAAGCAGAAUTAGCAG | Probe |
| SEQ ID NO: 56 | FB-O! | GCGGAGAAGGCAAAGCAGAAUTAGCAGTCTCCGC | Torch |
| SEQ ID NO: 57 | FB-O | GCGGAGAAGGCAAAGCAGAAUTAGCAG | Probe |
| SEQ ID NO: 58 | FB-P | TCTTTCCCACCRAACCAACA | Primer |
| SEQ ID NO: 59 | FB-Q | CTAGTTCTGCTTTGCCTTCTCCATCTTCT | Probe |
| SEQ ID NO: 60 | FB-R | AAGACTCCCACCGCAGTTTCAGCT | Probe |
| SEQ ID NO: 61 | FB-S | AAGACTCCCACCGCAGTTTCAGCT | Probe |
| SEQ ID NO: 62 | FB-T | AAGACTCCCACCGCAGTTTCAGCT | Probe |
| SEQ ID NO: 63 | FB-U | CTARTTCTGCTTTGCCTTCTCCATCTTCT | Probe |
| SEQ ID NO: 64 | FB-V | CTAGTTCTGCTTTGCCTTCTCCATCTTCT | Probe |
| SEQ ID NO: 65 | FB*-W | 3′-GAUUAAGACGAAACGGAAGAGGUAGAAGA-5′ | Probe |
| SEQ ID NO: 66 | FB*-X | 3′-GAUCAAGACGAAACGGAAGAGGUAGAAGA-5′ | Probe |
| SEQ ID NO: 67 | FB-Y | GAGACACAATTGCCTACCTGCTT | Primer |
| SEQ ID NO: 68 | FB-Z | GAGTCTAGGTCAAAUTCTTTCCCACC | Primer |
| SEQ ID NO: 69 | FB-AA | GGTGCTCTTGACCAAATTGGG | Primer |
| SEQ ID NO: 70 | FB-AB | CTTTCCCACCRAACCAACAGTG | Primer |
| SEQ ID NO: 71 | RA-A | TTAGTCATYACAGTGACTGACAACAAAGG | Probe |
| SEQ ID NO: 72 | RA-B | AGGTAAGCTCCWAGATCTACTAT | Primer |
| SEQ ID NO: 73 | RA-C | AGGTAAGCTCCWAGATCTACTAT | Primer |
| SEQ ID NO: 74 | RA-D | AGGTAAGCTCCTAGATCTACTAT | Primer |
| SEQ ID NO: 75 | RA-E | TTAGTCATUACAGTGACTGACAACAAAGGAGC | Probe |
| SEQ ID NO: 76 | RA-F | TAGACCATGTGAATTCCCTGC | Probe |
| SEQ ID NO: 77 | RA-G | TTAGTCATYACAGTGACTGACAACAAAGG | Probe |
| SEQ ID NO: 78 | RA-H | TTAGTCATUACAGTGACTGACAACAAAGG | Probe |
| SEQ ID NO: 79 | RA-I | ACAAATGCAAAAATCATACCTTACTC | Primer |
| SEQ ID NO: 80 | RA-J! | CGTGGCTTTATGTATTTGAATGCTCCTTTGGCCACG | Torch |
| SEQ ID NO: 81 | RA-J | CGTGGCTTTATGTATTTGAATGCTCCTTTG | Probe |
| SEQ ID NO: 82 | RA-K! | CGTGGCTTTATGTATTTGAATGCTCCTTTGGCCACG | Torch |
| SEQ ID NO: 83 | RA-K | CGTGGCTTTATGTATTTGAATGCTCCTTTG | Probe |
| SEQ ID NO: 84 | RA-L! | CGGTGGCTTTATGTATTTGAATGCTCCTTTGGCCACCG | Torch |
| SEQ ID NO: 85 | RA-L | CGGTGGCTTTATGTATTTGAATGCTCCTTTG | Probe |
| SEQ ID NO: 86 | RA-M! | CGGTGGCTTTATGTATTTGAATGCTCCTTTGAGCCACCG | Torch |
| SEQ ID NO: 87 | RA-M | CGGTGGCTTTATGTATTTGAATGCTCCTTTG | Probe |
| SEQ ID NO: 88 | RA-N | ACAAATGCAAAAATCATACCTTACTC | Primer |
| SEQ ID NO: 89 | RA-O | TTAGTCATTACAGTGACTGACAACAAAGG | Probe |
| SEQ ID NO: 90 | RA-P | TTAGTCATYACAGTGACTGACAACAAAGG | Probe |
| SEQ ID NO: 91 | RA-Q | TTAGTCATUACAGTGACTGACAACAAAGG | Probe |
| SEQ ID NO: 92 | RA-R | AGGTAAGCTCCGAGATCTACTAT | Primer |
| SEQ ID NO: 93 | RA-S | CTAGGTAAGCTCCAAGATCTACTAT | Primer |
| SEQ ID NO: 94 | RA-T | CTAGGTAAGCTCCAAGATCTACTAT | Primer |
| SEQ ID NO: 95 | RA-U | CTAGGTAAGCTCCTAGATCTACTAT | Primer |
| SEQ ID NO: 96 | RA-V | CTAGGTAAGCTCCTAGATCTACTAT | Primer |
| SEQ ID NO: 97 | RA*-W | 3′-AAUCAGUAGUGUCACUGACUGUUGUUUCC-5′ | Probe |
| SEQ ID NO: 98 | RA*-X | 3′-AAUCAGUAAUGUCACUGACUGUUGUUUCC-5′ | Probe |
| SEQ ID NO: 99 | RB-A | TGAAGTTGATGAACAAAGTGG | Primer |
| SEQ ID | RB-B | GATGATGATCCYGCATCACTAAC | Primer |
| NO: 100 | | | |
| SEQ ID | RB-C | GATGATGATCCUGCATCACTAAC | Primer |
| NO: 101 | | | |
| SEQ ID | RB-D | ATGGGTGCCTATGTTCCAGTCATCTG | Probe |
| NO: 102 | | | |
| SEQ ID | RB-E | CACCAGCCCTCAATACCACCC | Probe |
| NO: 103 | | | |
| SEQ ID | RB-F | GCTTCAATGGTCCACAGTT | Primer |
| NO: 104 | | | |
| SEQ ID | RB-G | GCTTCAATGGTCCACAGTT | Primer |
| NO: 105 | | | |
| SEQ ID | RB-U | GATGATGATCCUGCATCACTAAC | Primer |
| NO: 106 | | | |
| SEQ ID | RB-V! | CGCTGCTGGCACAGATGACTGGAACATAGCAGCG | Torch |
| NO: 107 | | | |
| SEQ ID | RB-V | CGCTGCTGGCACAGATGACTGGAACATA | Probe |
| NO: 108 | | | |
| SEQ ID | RB-W! | CCGAGCAAGTCTGCTGGCACAGATGACTGGGCTCGG | Torch |
| NO: 109 | | | |
| SEQ ID | RB-W | CCGAGCAAGTCTGCTGGCACAGATGACTGG | Probe |
| NO: 110 | | | |
| SEQ ID | RB-X! | CCGAGCAAGTCTGCTGGCACAGATGACTGGTTGCTCGG | Torch |
| NO: 111 | | | |
| SEQ ID | RB-X | CCGAGCAAGTCTGCTGGCACAGATGACTGG | Probe |
| NO: 112 | | | |
| SEQ ID | RB-Y! | CGCCAGTCATCTGTGCCAGCAGACTTGCTGGCG | Torch |
| NO: 113 | | | |
| SEQ ID | RB-Y | CGCCAGTCATCTGTGCCAGCAGACTTG | Probe |
| NO: 114 | | | |
| SEQ ID | RB-Z | TAGTATGTTGATGCTTGCAAGTTC | Primer |
| NO: 115 | | | |
| SEQ ID | Flu A | GenBank Accession No. KC355801.1 | Target |
| NO: 116 | | (13-JAN-13) | |
| SEQ ID | Flu B | GenBank Accession No. JX266956.1 | Target |
| NO: 117 | | (22-OCT-12) | |
| SEQ ID | RSV-A | GenBank Accession No. AY911262.1 | Target |
| NO: 118 | | (5-JUL-5) | |
| SEQ ID | RSV-B | GenBank Accession No. AF013254.1 | Target |
| NO: 119 | | (2-NOV-97 with non-sequence changes on | |
| | | 30-SEP-99) | |

*All sequences are written in the 5′ to 3′ orientation unless indicated otherwise. Sequence symbols are per Table 1 of World Intellectual Property Organization (WIPO) Handbook on Industrial Property Information and Documentation, Standard ST.25 (1998) (“WIPO ST.25 (1998)”). Sequence Names containing “!” are molecular torch probes. Bold/underline on a symbol indicates degenerate or non-Watson/Crick residue relative to target.

All of the articles, devices, systems, and methods disclosed and claimed herein can be made and executed without undue experimentation in light of the present disclosure. While the devices, systems, and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure. It will also be appreciated that computer-based embodiments of the instant disclosure can be implemented using any suitable hardware and software.

All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes.

What is claimed is:

1. A kit for analysis of a Respiratory Syncytial Virus B (RSV B) target nucleic acid molecule species that may be present in a biological sample, comprising:
an RSV B primer pair for generating an RSV B amplicon if RSV B is present in the biological sample, the RSV B primer pair comprising a first RSV B primer and a second RSV B primer, wherein:
(i) the first RSV B primer comprises a target hybridizing sequence consisting of a nucleic acid sequence selected from the group consisting of: SEQ ID NOs:99-101 and 106; and
(ii) the second RSV B primer comprises a target hybridizing sequence consisting of a nucleic acid sequence selected from the group consisting of SEQ ID NOs:104-106 and 115.

2. The kit of claim 1, wherein the first and/or second RSV A primer comprises a primer upstream region having a nucleotide sequence that is not complementary to a sequence present in the RSV A target nucleotide sequence.

3. The kit of claim 1, further comprising a RSV B probe molecule species that is substantially complementary to an RSV probe target nucleic acid sequence, is about 21 to about 100 contiguous bases in length and comprises an oligonucleotide sequence selected from the group consisting of SEQ ID NOS: 102, 103, and 107-114.

4. The kit of claim 3, wherein the RSV B probe molecule species is up to about 60 nucleotide residues in length and further comprises a non-target hybridizing sequence.

5. The kit of claim 3, wherein the RAV B probe molecule species comprises a detectable label.

6. The kit of claim 5, wherein the detectable label is selected from the group consisting of: a chemiluminescent moiety, a fluorophore moiety, a quencher moiety, and both a fluorophore moiety and a quencher moiety.

7. The kit of claim 5, wherein the detectable label comprises a donor/acceptor label pair.

8. The kit of claim 6, further comprising one or more of a first Flu A primer pair, a second Flu A primer pair, a Flu B primer pair, and an RSV A primer pair.

9. The kit of claim 8, further comprising a probe molecule species for detecting an amplification product of each of the first Flu A primer pair, second Flu A primer pair, Flu B primer pair, and RSV A primer pair present in the kit, wherein each probe molecule species in the kit is distinguishably labeled such that any one probe molecule species can be distinguished from the other probe molecules species in the kit.

10. A reaction mixture comprising the first RSV B primer, the second RSV B primer, and the RSV B probe molecule species of claim 3, and further comprising one or more of a reverse transcriptase, a DNA polymerase, a buffer, and dNTPs.

11. A method of determining whether a biological sample contains RSV B, comprising:
(a) contacting, under stringent hybridization conditions, nucleic acid molecules from the biological sample with a labelled RSV B probe molecule species that is about 21 to about 100 contiguous bases in length, and comprises an oligonucleotide sequence selected from the group consisting of SEQ ID NOS: 102, 103, and 107-114, wherein if a RSV B is present in the biological sample then a RSV B nucleic acid molecule in the sample forms a probe:target duplex with the labeled RSV A probe molecule species; and
(b) detecting the presence of absence of the probe:target duplex;
wherein detecting the presence of the probe:target duplex indicates the presence of RSV B in the sample.

12. The method of claim 11, wherein the method further comprises, prior to step (a):
(i) contacting the biological sample with a RSV A primer pair comprising a first RSV A primer comprising a target hybridizing sequence consisting of the nucleic acid sequence of SEQ ID NO:79, and a second RSV A primer comprising a target hybridizing sequence consisting of the nucleic acid sequence of SEQ ID NO:92;
(ii) performing a nucleic acid amplification reaction, wherein the RSV A nucleic acid molecule, if present in the biological sample, is amplified.

13. A composition comprising:
(a) an RSV B primer pair for generating an RSV B amplicon if RSV B is present in the biological sample, the RSV B primer pair comprising a first RSV B primer and a second RSV B primer, wherein:
(i) the first RSV B primer comprises a target hybridizing sequence consisting of a nucleic acid sequence selected from the group consisting of: SEQ ID NOs:99-101 and 106; and
(ii) the second RSV B primer comprises a target hybridizing sequence consisting of a nucleic acid sequence selected from the group consisting of: SEQ ID NOs:104-106 and 115;
(b) a probe molecule species that is substantially complementary to an RSV B probe comprising a target hybridizing sequence consisting of a nucleic acid sequence selected from the group consisting of: SEQ ID NOs: 102, 103, and 107-114; and
(c) an RSV B amplicon, which amplicon is generated by a nucleic acid amplification process that utilizes the RSV B primer pair.

14. A method for the determining the presence or absence of a RSV B target nucleic acid in a sample, the method comprising the steps of:
(A) contacting the RSV B primer pair of claim 1;
(B) performing an in vitro nucleic acid amplification reaction wherein the RSV B target nucleic acid, if present in the sample, is used by the RSV B primer pair to amplify the RVS B target nucleic acid to generate an amplification product; and
(C) detecting the amplification product;
thereby determining the presence or absence of the target nucleic acid in the sample.

15. The method of claim 14, wherein the detecting step (C) is performed using a detectably labelled RSV B probe molecule species, wherein the RSV B probe molecule species is substantially complementary to an RSV B probe target nucleic acid sequence, is about 21 to about 100 contiguous bases in length and comprises an oligonucleotide sequence selected from the group consisting of: SEQ ID NOs: 102, 103, and 107-114.

16. A reaction mixture comprising:
(a) a first RSV B primer comprising a target hybridizing sequence consisting of a nucleic acid sequence selected from the group consisting of: SEQ ID NOs:99-101 and 106; and
(b) a second RSV B primer comprising a target hybridizing sequence consisting of a nucleic acid sequence selected from the group consisting of SEQ ID NOs: 104-106 and 115.

17. The reaction mixture of claim 16, further comprising: a RSV B probe molecule species that is substantially complementary to an RSV probe target nucleic acid sequence, is about 21 to about 100 contiguous bases in length and comprises an oligonucleotide sequence selected from the group consisting of SEQ ID NOS: 102, 103, and 107-114.
